(19) United States
(12) Patent Application Publication
(10) Pub. No.: US 2023/0114016 A1
(43) Pub. Date: Apr. 13, 2023

(54) DISPLAY DEVICE

(71) Applicant: SAMSUNG DISPLAY CO.,LTD., YONGIN-SI, GYEONGGI-DO (KR)

(72) Inventors: Bo Ram CHOI, ASAN-SI (KR); Gyeong Ub MOON, SUWON-SI (KR); Jong Yeop AN, HWASEONG-SI (KR); Hyeon Jun LEE, SEOUL (KR)

(21) Appl. No.: 17/804,755

(22) Filed: May 31, 2022

(30) Foreign Application Priority Data
Oct. 13, 2021 (KR) 10-2021-0135687

(51) Int. Cl. A61B 5/024; H01L 27/32; H01L 51/00; A61B 5/021

(52) U.S. Cl. CPC A61B 5/02427; H01L 27/3234; H01L 27/3244; H01L 51/0097; A61B 5/02108; H01L 2251/5338; G06F 3/0443

(57) ABSTRACT

A display device measures a user's blood pressure by detecting a photoplethysmography signal. A display device includes a display panel that includes a through hole and a pixel area that surrounds the through hole and includes pixels that display an image, a pressure sensor disposed on a surface of the display panel and configured to sense an externally applied pressure, a light-emitting member disposed at a position that overlaps the through hole of the display panel and outputs light toward a front side of the display panel through the through hole, and a light-receiving sensor that faces the front side of the display panel and is configured to sense light reflected from the front side of the display panel toward the display panel.

### CROSS-REFERENCE TO RELATED APPLICATIONS

This application claims priority under 35 U.S.C. 119 from Korean Patent Application No. 10-2021-0135687, filed on Oct. 13, 2021 in the Korean Intellectual Property Office, the contents of which are herein incorporated by reference in their entirety.

### 1. TECHNICAL FIELD

Embodiments of the present disclosure are directed to a display device.

### 2. DISCUSSION OF THE RELATED ART

A display device displays images on a screen and is used not only by TVs and monitors but also by portable smart phones, tablet PCs, etc. A portable display device has a variety of features in addition to that of displaying images. For example, a camera and a fingerprint sensor may be equipped in a display device.

Recently, methods for more conveniently acquiring biometric information on health are being developed. For example, a traditional oscillometric blood pressure measurement device may be incorporated into a portable blood pressure measurement device. A portable blood pressure measurement device itself requires a separate light source, a sensor and a display, and would be carried by a user in addition to a portable smart phone or tablet PC, which is inconvenient.

### SUMMARY

Embodiments of the present disclosure provide a display device capable of measuring a users blood pressure by detecting a photoplethysmography signal.

Embodiments of the present disclosure also provide a display device that can reduce a path for detecting a pulse wave signal for blood pressure measurement.

According to an embodiment of the disclosure, a display device comprises a display panel that includes a through hole and a pixel area that surrounds the through hole and includes pixels that display an image, a pressure sensor disposed on a surface of the display panel and configured to sense an externally applied pressure, a light-emitting member disposed at a position that overlaps the through hole of the display panel and outputs light through the through hole toward a front side of the display panel, and a light-receiving sensor that faces the front side of the display panel and is configured to sense light reflected from the front side of the display panel toward the display panel.

In an embodiment, the pressure sensor includes a first optical hole that overlaps the through hole in a thickness direction of the display panel.

In an embodiment, a dead space area surrounds the through hole and the light-receiving sensor is disposed in the dead space area adjacent to the through hole of the display panel wherein the light-receiving sensor senses light reflected by a body part or an object from the front side of the display panel.

In an embodiment, the display device further comprising a panel bottom cover that includes a second optical hole located on a rear side of the pressure sensor and aligned with the through hole of the display panel in the thickness direction of the display panel, wherein the panel bottom cover protects the display panel.

In an embodiment, the display device further comprising a main circuit board. The main circuit board fixes the light-emitting member so that the light-emitting member is aligned with the through hole of the display panel, and a main processor is mounted on the main circuit board and is configured to generate a pulse wave signal based on an optical signal according to an amount of light detected by the light-receiving sensor and calculate a blood pressure based on the pulse wave signal.

In an embodiment, light emitted from the light-emitting emitting member is absorbed by or reflected from a blood vessel of a user's finger or wrist through the first and second optical holes and the through hole. The reflected light is sensed by the light-receiving sensor.

In an embodiment, the light-emitting member comprises at least one of: a first light-emitting diode that emits infrared light; a second light-emitting diode that emits green light; and a third light-emitting diode that emits visible light of a color other than the green light.

In an embodiment, the light-emitting member comprises two or more of: a first light-emitting diode that emits infrared light; a second light-emitting diode that emits green light; and a third light-emitting diode that emits light of a color other than the green light.

In an embodiment, the main processor controls the plurality of light-emitting diodes in the light-emitting member and simultaneously turns on or turns off the plurality of light-emitting diodes at a predetermined driving period, or sequentially turns on or turns off the plurality of light-emitting diodes at different periods.

In an embodiment, the main processor controls the plurality of light-emitting diodes in the light-emitting member so that at least two of the plurality of light-emitting diodes are simultaneously turned on or turned off at a predetermined first driving period that the other of the plurality of light-emitting diodes is sequentially turned on or turned off in a different period from that of the first time period.

In an embodiment, the pressure sensor comprises: a first base substrate and a second base substrate that face each other; a first pressure sensor electrode disposed on the first base substrate; a second pressure sensor electrode disposed on the second base substrate; and a pressure sensing layer that overlaps the first and second pressure sensor electrodes in a thickness direction of the first base substrate.

In an embodiment, the first and second pressure sensor electrodes include a transparent conductive material, and the pressure sensing layer includes a transparent polymer resin so that the pressure sensor transmits light.

According to an embodiment of the disclosure, a display device comprises a display panel that includes a transparent region and a pixel area that surrounds the transparent region and includes pixels that display an image, a pressure sensor disposed on a surface of the display panel and configured to sense an externally applied pressure, a light-emitting member disposed at a position that overlaps the transparent region of the display panel and outputs light through the transparent region toward a front side of the display panel; and a light-receiving sensor that faces the front side of the display panel and is configured to sense light reflected from the front side of the display panel toward an inside of the transparent region.

In an embodiment, the pressure sensor includes a first optical hole that overlaps the transparent region in a thickness direction of the display panel.

In an embodiment, a dead space area surrounds the through hole and the light-receiving sensor is formed in the dead space area adjacent to the transparent region of the display panel. The light-receiving sensor senses light that is reflected by a body part or an object on a front side of the transparent region and is incident toward the transparent region.

In an embodiment, the display device further comprises a bracket disposed on a rear side of the pressure sensor and that includes a sensor hole that is aligned with the transparent region of the display panel in the thickness direction of the display panel.

In an embodiment, the light-receiving sensor is fixed on a main circuit board that fixes the light-emitting member so that the light-emitting member is aligned with the through hole of the display panel, and is disposed in the sensor hole of the bracket.

In an embodiment, the display device further comprises a panel bottom cover disposed on a rear side of the pressure sensor and that includes a second optical hole that is aligned with the through hole of the display panel in the thickness direction of the display panel, wherein the panel bottom cover protects the display panel.

In an embodiment, the light-receiving sensor is fixed on a main circuit board that fixes the light-emitting member so that the light-emitting member is aligned with the through hole of the display panel, and is disposed in the second optical hole of the panel bottom cover.

In an embodiment, the display device further comprises a cover window disposed on the front side of the display panel, and the light-receiving sensor is integrally formed with or incorporated into the cover window.

According to an embodiment of the disclosure, a method of measuring blood pressure by a display device includes recognizing, by a touch sensitive display device, a pressure applied to the display device by an object; detecting the pressure applied to the display device by the object as the pressure increases to a maximum value and then decreases using a pressure sensor; sensing light reflected by the object using a light-receiving sensor; generating a pulse wave signal according to the pressure based on a pressure value calculated by the pressure sensor and an optical signal according to an amount of light sensed by the light-receiving sensor, and calculating a blood pressure based on the pulse wave signal.

In an embodiment, the object is a part of a user's body.

In an embodiment, the method further comprises operating the display device in a blood pressure measurement mode, after recognizing that a touch is made. When a user touches a position that is not a blood pressure measurement position, the display device operates in a touch mode, and when the user touches a position associated with a blood pressure measurement position, the display device operates in the blood pressure measurement mode.

According to embodiments of the present disclosure, when light emitted from a light-emitting member is reflected by a user's body part such as a finger blood vessel, the reflected light is sensed by a light-receiving sensor of a display panel. Accordingly, the user's blood pressure can be calculated based on the amount of light sensed by the light-receiving sensor and a pressure sensed by a pressure sensor.

In a display device according to an embodiment of the present disclosure, the light from the light-emitting member is reflected from a user's body part through a pressure sensor and a transparent region that is an optical hole of a display panel. In addition, the reflected light is sensed by the light-receiving sensor of the display panel, and thus the path and distance of the light reflected from a user's body part can be reduced, which increases the accuracy and reliability of the signal detection.

### BRIEF DESCRIPTION OF THE DRAWINGS

FIG. 1 is a perspective view of a display device according to an embodiment of the present disclosure.

FIG. 2 is an exploded, perspective view of a display device according to an embodiment of the present disclosure.

FIG. 3 is a plan view of a display panel, a display circuit board, a display driver circuit and a touch driver circuit according to an embodiment of the present disclosure.

FIG. 4 illustrates how to measure blood pressure by a display device according to an embodiment.

FIG. 5 is a flowchart of a method of measuring blood pressure by a display device according to an embodiment.

FIG. 6 is a cross-sectional view of the structure of a cover window, a display panel, a pressure sensor, a light-emitting member and a light-receiving sensor, taken along line I-I′ of FIG. 4.

FIG. 7 is a graph for calculating blood pressure by a main processor according to an embodiment of the present disclosure.

FIG. 8 illustrates a layout of a display area and a through hole of a display panel according to an embodiment.

FIG. 9 is a cross-sectional view of a display panel, taken along line II-II′ of FIG. 8.

FIG. 10 illustrates a layout of pressure sensor electrodes of a pressure sensor and a first optical hole according to an embodiment of the present disclosure.

FIG. 11 is a cross-sectional view of a pressure sensor of FIG. 10.

FIG. 12 is another cross-sectional view of a cover window, a display panel, a pressure sensor, a light-emitting member and a light-receiving sensor, taken along line I-I′ of FIG. 4.

FIG. 13 is another cross-sectional view of a cover window, a display panel, a pressure sensor, a light-emitting member and a light-receiving sensor, taken along line I-I′ of FIG. 4.

FIG. 14 is a timing diagram of driving control signals for a light-emitting member shown in FIG. 13.

FIG. 15 is a graph for calculating blood pressure by using a light-emitting member shown in FIG. 13.

FIG. 16 is a cross-sectional view of a cover window, a display panel, a pressure sensor, a light-emitting member and a light-receiving sensor according to an embodiment, taken along line I-I′ of FIG. 4.

FIG. 17 is a cross-sectional view of a cover window, a display panel, a pressure sensor, a light-emitting member and a light-receiving sensor according to an embodiment, taken along line I-I′ of FIG. 4.

FIG. 18 is a cross-sectional view of a cover window, a display panel, a pressure sensor, a light-emitting member and a light-receiving sensor according to an embodiment, taken along line I-I′ of FIG. 4.

FIG. 19 illustrates a layout of pressure sensor electrodes of a light-emitting member shown in FIG. 18.

FIGS. 20 and 21 are perspective views of a display device according to an embodiment.

FIGS. 22 and 23 are perspective views of a display device according to an embodiment of the present disclosure.

### DETAILED DESCRIPTION

Embodiments of the present disclosure will now be described more fully hereinafter with reference to the accompanying drawings, in which embodiments of the disclosure are shown.

It will be understood that when a layer is referred to as being “on” another layer or substrate, it can be directly on the other layer or substrate, or intervening layers may also be present. The same reference numbers may indicate the same components throughout the specification.

Hereinafter, embodiments of the present disclosure will be described with reference to the accompanying drawings.

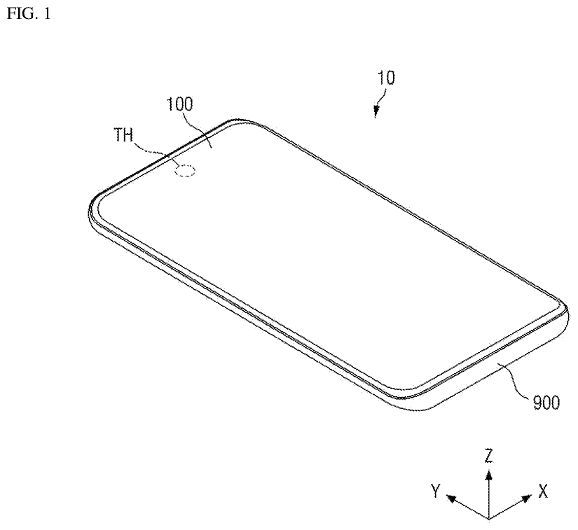

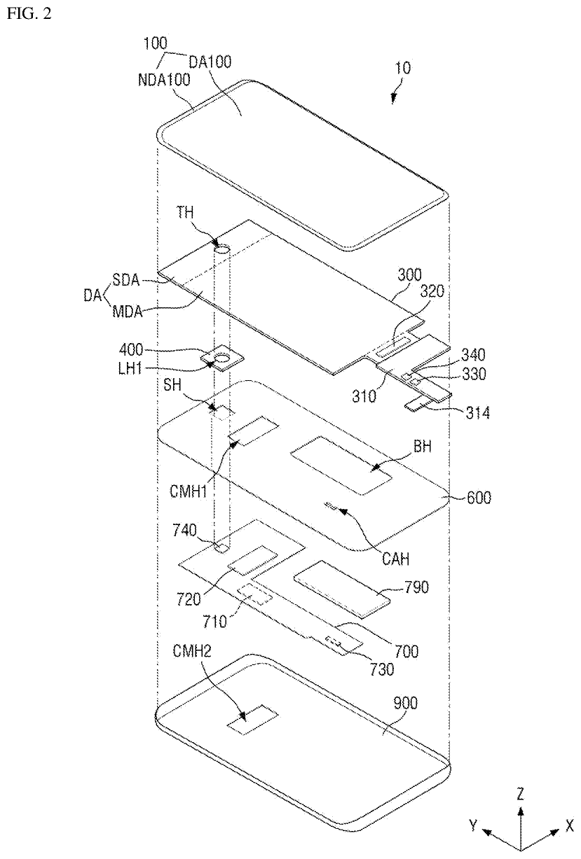

FIG. 1 is a perspective view of a display device according to an embodiment of the present disclosure. FIG. 2 is an exploded, perspective view of a display device according to an embodiment of the present disclosure.

Referring to FIGS. 1 and 2, a display device 10 according to an embodiment of the present disclosure may be used by a portable electronic device such as a mobile phone, a smart phone, a tablet PC, a mobile communications terminal, an electronic notebook, an electronic book, a portable multimedia player (PMP), a navigation device or an ultra mobile PC (UMPC). Alternatively, the display device 10 according to an embodiment of the present disclosure may be used as a display unit of a television, a laptop computer, a monitor, an electronic billboard, or an Internet of Things (IOT) device. Alternatively, the display device 10 according to an embodiment of the present disclosure may be used by a wearable device such as a smart watch, a watch phone, a glasses-type display, or a head-mounted display (HMD) device. Alternatively, the display device 10 according to an embodiment may be used as a central information display (CID) disposed at an instrument cluster, a central fascia or a dashboard of a vehicle, as a room mirror display for the side mirrors of a vehicle, or as a display placed on the back of the front seats of a vehicle as an entertainment system for passengers in the rear seats thereof.

As used herein, the first direction (x-axis direction) is the shorter side direction of the display device 10, such as the horizontal direction of the display device 10. The second direction (y-axis direction) is the longer side direction of the display device 10, such as the vertical direction of the display device 10. A third direction (z-axis direction) is the thickness direction of the display device 10.

The display device 10 may have a substantially rectangular shape when viewed from the top. For example, the display device 10 may have shorter sides in a first direction (x-axis direction) and longer sides in a second direction (y-axis direction) when viewed from the top as shown in FIG. 1. Each of the corners where the short side in the first direction (x-axis direction) meets the longer side in the second direction (y-axis direction) may be rounded with a predetermined curvature or may be a right angle. The shape of the display device 10 when viewed from the top is not necessarily limited to a rectangular shape, but may have a polygonal shape, a circular shape, or an elliptical shape.

The display device 10 may be flat. Alternatively, the display device 10 may have two sides facing each other that are bent. For example, the left and right sides of the display device 10 are bent. Alternatively, each of the upper side, the lower side, the left side and the right side of the display device 10 are bent.

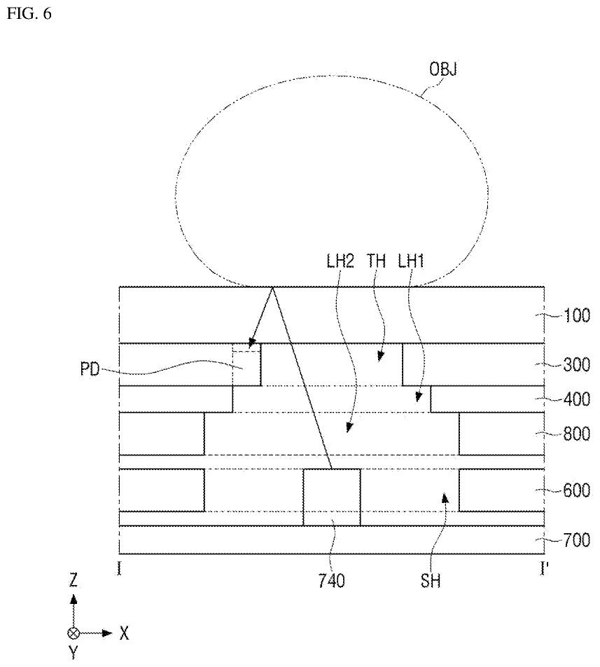

The display device 10 according to an embodiment of the present disclosure includes a cover window 100, a display panel 300, a light-receiving sensor PD (see FIG. 6) disposed in the display panel 300, a display circuit board 310, a display driver circuit 320, a pressure sensor 400, a bracket 600, a main circuit board 700, a main processor 710, a light-emitting member 740, a bottom cover 900.

The cover window 100 is disposed on the display panel 300 and covers the front surface of the display panel 300. Thus, the cover window 100 protects the front surface of the display panel 300.

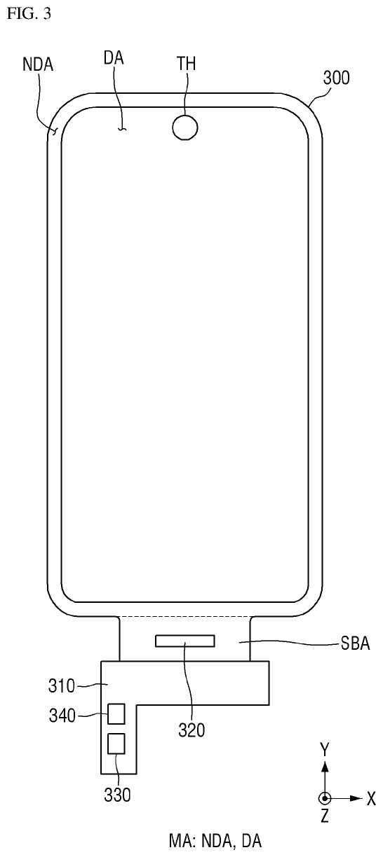

The cover window 100 includes a transmissive area DA100 that corresponds to the display panel 300 and a non-transmissive area NDA100 (see FIG. 3) that corresponds to an area other than the display panel 300. The non-transmissive area NDA 100 is opaque. Alternatively, the non-transmissive area NDA 100 is a decorative layer that has a pattern that can be displayed to a user when no image is displayed.

The display panel 300 is disposed under the cover window 100. The display panel 300 includes a display area DA and a non-display area NDA. The display area DA includes pixels that display images. The non-display area NDA does not display images and is disposed around the display area NDA. The non-display area NDA has no pixels. The non-display area NDA surrounds the display area DA as shown in FIG. 2, but embodiments of the present disclosure are not necessarily limited thereto. The display area DA occupies most of the area of the display panel 300.

The display panel 300 includes a through hole TH. The through hole TH penetrates through the display panel 300. The through hole TH is surrounded by the display area DA. The through hole TH is aligned in the third direction (z-axis direction) with a location of the main circuit board 700 where the light-emitting member 740 is attached and a sensor hole SH of the bracket 600. Accordingly, light emitted from the light-emitting member 740 of the main circuit board 700 passes through the sensor hole SH of the bracket 600 and the through hole TH of the display panel 300 to exit toward the front side of the display panel 300.

At least one light-receiving sensor PD that faces the front side is disposed around the through hole TH of the display panel 300, i.e., the periphery of the through hole TH. Therefore, light that is emitted from the light-emitting emitting member 740 and reflected from a body part or an object placed above the through hole TH may be incident on at least one light-receiving sensor PD disposed on the periphery of the through hole TH. Accordingly, even though at least one light-receiving sensor PD is disposed such that it faces the front side of the display panel 300, it can sense light reflected and incident on the front side of the display device 10.

Although FIG. 2 shows the display panel 300 as including one through hole TH, the number of through holes TH is not necessarily limited thereto. When the display panel 300 includes a plurality of through holes TH, one of the plurality of through holes TH may overlap the light-emitting member 740 in the third direction (z-axis direction), and the others of the plurality of through holes TH may overlap a sensor device other than the light-emitting member 740. For example, the sensor device may be a proximity sensor, an illuminance sensor, or a front camera sensor.

In an embodiment, the display panel 300 is a light-emitting display panel that includes light-emitting elements. For example, the display panel 300 is an organic light-emitting display panel that uses organic light-emitting diodes that include an organic emissive layer, a micro light-emitting diode display panel that uses micro LEDs, a quantum-dot light-emitting display panel that uses quantum-dot light-emitting diodes that include an quantum-dot emissive layer, or an inorganic light-emitting display panel that uses inorganic light-emitting elements that include an inorganic semiconductor. In the following description, the display panel 300 is an organic light-emitting display panel.

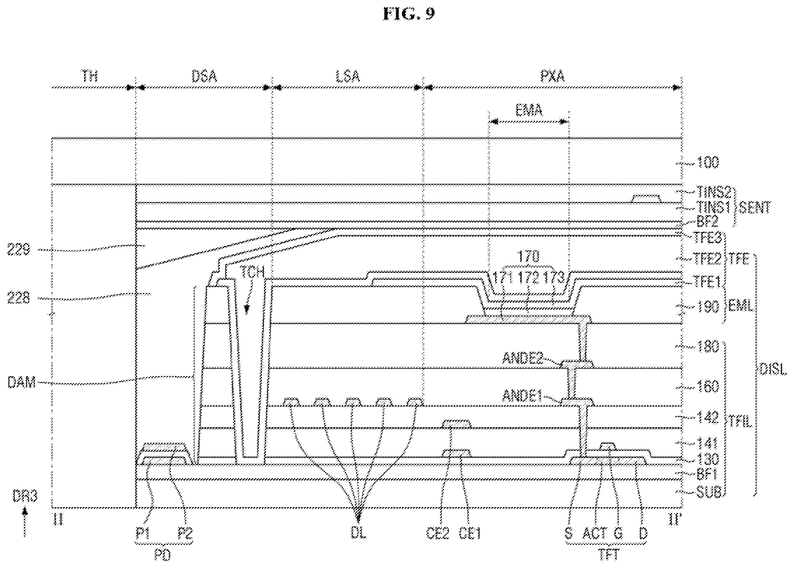

In addition, the display panel 300 includes a touch electrode layer that includes touch electrodes that sense an object such as a person's finger or a pen. The touch electrode layer is disposed on a display layer that includes pixels that display images. The display layer and the touch electrode layer will be described in detail below with reference to FIG. 9 et seq.

The display circuit board 310 and the display driver circuit 320 are attached to one side of the display panel 300. The display circuit board 310 may be a flexible printed circuit board that can be bent, a rigid printed circuit board that is rigid and not bendable, or a hybrid printed circuit board that includes a rigid printed circuit board and a flexible printed circuit board.

The display driver circuit 320 receives control signals and supply voltages through the display circuit board 310 and generates and outputs signals and voltages that drive the display panel 300. In an embodiment, display driver circuit 320 is implemented as an integrated circuit (IC) and is attached to the display panel 300 by a chip on glass (COG) technique, a chip on plastic (COP) technique, or an ultrasonic bonding. However, embodiments of the present disclosure are not necessarily limited thereto. In an embodiment, the display driver circuit 320 is attached on the display circuit board 310.

A touch driver circuit 330 and a pressure driver circuit 340 are disposed on the display circuit board 310. In an embodiment, each of the touch driver circuit 330 and the pressure driver circuit 340 is implemented as an integrated circuit and is attached to the upper surface of the display circuit board 310. Alternatively, in an embodiment, the touch driver circuit 330 and the pressure driver circuit 340 are implemented as a single integrated circuit.

The touch driver circuit 330 is electrically connected to touch electrodes of the touch electrode layer of the display panel 300 through the display circuit board 310. The touch driver circuit 330 outputs touch driving signals to the touch electrodes and senses a capacitance voltage of the touch electrodes.

The touch driver circuit 330 generates touch data based on a change in the electric signal sensed by each of the touch electrodes and transmits the touch data to the main processor 710. The main processor 710 analyzes the touch data and calculates the coordinates of the position that received the touch input. Touches include a physical contact or a near proximity. A physical contact refers to when an object such as the user's finger or a pen is brought into contact with the cover window on the touch electrode layer. A near proximity refers to when an object such as a person's finger or a pen is brought close to the cover window 100 without touching it, such as hovering over it.

The pressure sensor 400 senses an externally applied pressure and generates an electrical signal in response to detecting an applied pressure. The pressure driver circuit 340 senses the electrical signal received from a pressure sensor electrode of the pressure sensor 400, converts the sensed signal into pressure data, and transmit it to the main processor 710. The main processor 710 determines whether pressure is being applied to the pressure sensor 400 and if so, calculates the amount of the pressure being applied to the pressure sensor 400 based on the pressure data.

A power supply unit that supplies display driving voltages that drive the display driver circuit 320 is further disposed on the display circuit board 310.

The bracket 600 is disposed under the display panel 300. The bracket 600 may include plastic, metal, or both plastic and metal. In the bracket 600, there are formed a first camera hole CMH1 in which a first camera sensor 720 is inserted, a battery hole BH in which a battery is disposed, a cable hole CAH through which a cable 314 connected to the display circuit board 310 passes, and the sense hole SH that overlaps in the third direction (z-axis direction) with the location where the light-emitting member 740 is attached. The light-emitting member 740 is disposed in the sensor hole SH. Alternatively, the bracket 600 does not include the sensor hole SH.

The main circuit board 700 and a battery 790 are disposed under the bracket 600. The main circuit board 700 may be either a printed circuit board or a flexible printed circuit board.

The main circuit board 700 includes the main processor 710, the first camera sensor 720, a main connector 730 and the light-emitting member 740. The first camera sensor 720 is disposed on both the upper and lower surfaces of the main circuit board 700, the main processor 710 is disposed on the upper surface of the main circuit board 700, and the main connector 730 is disposed on the lower surface of the main circuit board 700. The light-emitting member 740 is disposed on the upper surface of the display circuit board 700.

The main processor 710 controls all functions of the display device 10. For example, the main processor 710 outputs digital video data to the display driver circuit 320 through the display circuit board 310 so that the display panel 300 can display images. In addition, the main processor 710 receives touch data from the touch driver circuit 330 to determine the coordinates of a user's touch, and then executes an application indicated by an icon displayed at the coordinates of the user's touch. In addition, the main processor 710 converts first image data received from the first camera sensor 720 into digital video data and outputs it to the display driver circuit 320 through the display circuit board 310, so that an image captured by the first camera sensor 720 is displayed on the display panel 300. In addition, the main processor 710 check a user's blood pressure based on the sensor signal input from the light-receiving sensor PD.

The first camera sensor 720 processes image frames such as still images and video data obtained by an image sensor and outputs them to the main processor 710. The first camera sensor 720 may be a CMOS image sensor or a CCD sensor. The first camera sensor 720 is exposed to the lower surface of the bottom cover 900 through a second camera hole CMH2, and thus can capture an image of an object or a background under the display device 10.

The cable 314 that passes through the cable hole CAH of the bracket 60 is connected to main connector 730. Accordingly, the main circuit board 700 is electrically connected to the display circuit board 310.

The light-emitting member 740 includes a light source that emits light in the visible or infrared wavelength range. The light source includes, for example, at least one of a light-emitting diode (LED), an organic light-emitting diode (OLED), a laser diode (LD), a quantum-dot (QD) light-emitting diode, or a phosphor.

The light-receiving sensor PD disposed or formed adjacent to the through hole TH of the display panel 300 senses light reflected and incident toward the through hole TH. The light-receiving sensor PD may be a photodiode or a phototransistor. For example, the light-receiving sensor PD may be a CMOS image sensor or CCD sensor that can detect light. The light-receiving sensor PD outputs an optical signal to the main processor 710 in proportion to the amount of light reflected off an object above the through hole TH. The emission by the light-emitting member 740 and the structure of the light-receiving sensor PD will be described below in more detail with reference to the accompanying drawings.

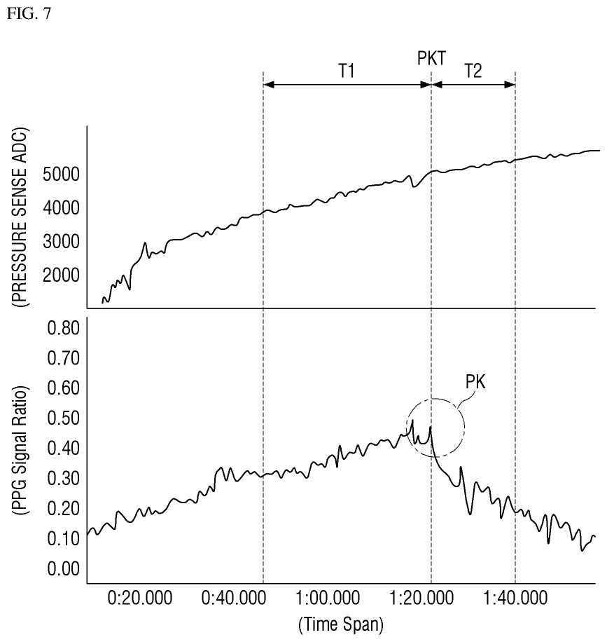

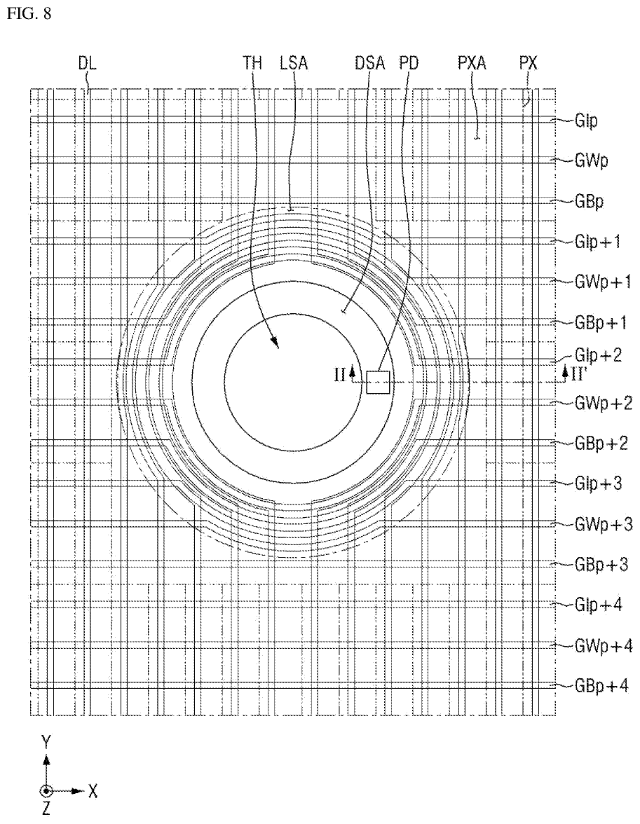

The main processor 710 calculates a pulse wave signal that reflects changes in the blood according to a heartbeat based on an optical signal received from the light-receiving sensor PD. The main processor 710 determines the user's blood pressure based on the pulse wave signal. A method of measuring a person's blood pressure using the light-receiving sensor PD will be described below with reference to FIGS. 7 and 8.

The battery 790 is disposed so that it does not overlap the main circuit board 700 in the third direction (z-axis direction). The battery 790 overlaps the battery hole BH of the bracket 600.

In addition, a mobile communications module that can transmit or receive a radio signal to or from at least one of a base station, an external terminal or a server over a mobile communications network is further mounted on the main circuit board 700. The wireless signal may be one of various types of data, such as a voice signal, a video call signal, or a text/multimedia message transmission/reception.

The bottom cover 900 is disposed under the main circuit board 700 and the battery 790. The bottom cover 900 is fastened and fixed to the bracket 600. The bottom cover 900 forms the exterior of the lower surface of the display device 10. The bottom cover 900 may include plastic, metal or plastic and metal.

The second camera hole CMH2 is formed in the bottom cover 900, through which the lower surface of the first camera sensor 720 is exposed. The position of the first camera sensor 720 and the positions of the first and second camera holes CMH1 and CMH2 that are aligned with the first camera sensor 720 are not limited to those of an embodiment shown in FIG. 2.

FIG. 3 is a plan view of a display panel, a display circuit board, a display driver circuit and a touch driver circuit according to an embodiment of the present disclosure.

Referring to FIG. 3, the display panel 300 may be a rigid display panel that is rigid and thus is not easily bent, or a flexible display panel that is flexible and thus can be easily bent, folded or rolled. For example, the display panel 300 may be a foldable display panel that can be folded and unfolded, a curved display panel that has a curved display surface, a bent display panel that has a bent area other than the display surface, a rollable display panel that can be rolled and unrolled, or a stretchable display panel that can be stretched.

In addition, in an embodiment, the display panel 300 is implemented as a transparent display panel that allows a user to see an object or a background behind the display panel from the front side of the display panel 300. In addition, in an embodiment, the display panel 300 is implemented as a reflective display panel that can reflect an object or a background on the front side of the display panel 300.

The display panel 300 includes a main area MA and a subsidiary area SBA at one side of the main area MA. The main area MA includes a display area DA in which images are displayed, and a non-display area NDA around the display area DA. The display area DA occupies most of the main area MA. The display area DA is disposed at the center of the main area MA. The non-display area NDA is disposed on the outer side of the display area DA. The non-display area NDA is an edge of the display panel 300. In an embodiment, the non-display area NDA surrounds the display area DA.

Although FIG. 3 shows that the through hole TH of the display panel 300 is a physical hole, embodiments of the present disclosure are not necessarily limited thereto. In an embodiment, through hole TH is an optical hole through which light can pass. Alternatively, in an embodiment, the through hole TH is a combination of a physical hole and an optical hole.

Since the through hole TH overlaps the light-emitting member 740 in the third direction (z-axis direction) as shown in FIG. 2, light that passes through the through hole TH exits through the front side the display panel 300. If a part of the user's body or an object is placed in front of the display panel 300 in line with the through hole TH, the light of the light-emitting member 740 that passes through the through hole TH is reflected from the part of the body or the object and is incident on the front side of the through hole TH. Accordingly, the light-receiving sensor PD senses light incident on the front side of the display device 10 even though the light-receiving sensor PD is disposed on the front side of the display panel 300. For example, the light-receiving sensor PD senses light reflected by the object placed on the through hole TH.

In an embodiment, the through hole TH is surrounded by the display area DA. Alternatively, in an embodiment, the through hole TH is surrounded by the non-display area NDA or is disposed between the display area DA and the non-display area NDA. In addition, although FIG. 3 shows the through hole TH as being formed at the upper center of the display panel 300, the position of the through hole TH is not limited thereto.

The subsidiary area SBA protrudes from one side of the main area MA in the second direction (y-axis direction). As shown in FIG. 2, the length of the subsidiary area SBA in the first direction (x-axis direction) is less than the length of the main area MA in the first direction (x-axis direction). The length of the subsidiary area SBA in the second direction (y-axis direction) is less than the length of the main area MA in the second direction (y-axis direction). However, embodiments of the present disclosure are not necessarily limited thereto. In some embodiments, the sub-area SBA may be bent and may be disposed under the display panel 300, and may overlap the main area MA in the thickness direction (z-axis direction).

In an embodiment, the subsidiary area SBA of the display panel 300 can be bent so that it is located under the display panel 300. When bent, the subsidiary area SBA of the display panel 300 overlaps the main area MA of the display panel 300 in the third direction (z-axis direction).

The display circuit board 310 and the display driver circuit 320 are attached to the subsidiary area SBA of the display panel 300. The display circuit board 310 is attached to pads of the subsidiary area SBA of the display panel 100 using a low-resistance, high-reliability material such as an anisotropic conductive layer or a self assembly anisotropic conductive paste (SAP). The touch driver circuit 330 and the pressure driver circuit 340 are disposed on the display circuit board 310.

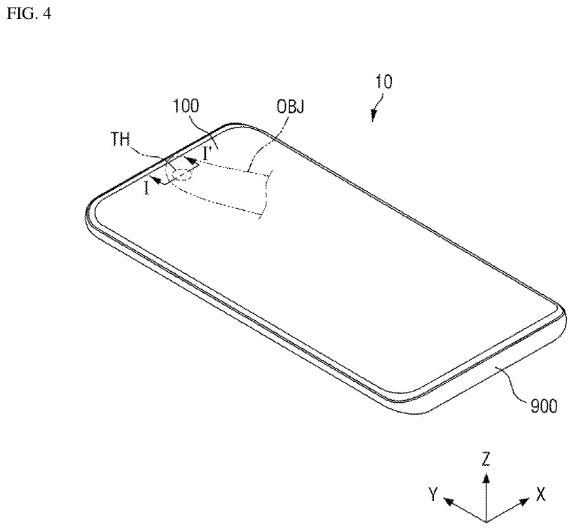

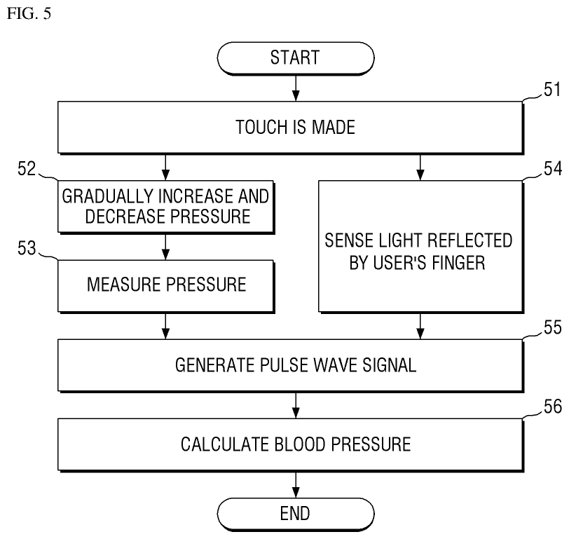

FIG. 4 illustrates how to measure blood pressure by a display device according to an embodiment. FIG. 5 is a flowchart of a method of measuring blood pressure by a display device according to an embodiment.

Referring to FIGS. 4 and 5, in an embodiment, when a part of the user's body, such as a finger OBJ, touches the front surface of the display device 10, the display device 10 recognizes that a touch is made (step 51). The display device 10 recognize a user's touch by using the touch electrode layer of the display panel 300 or the pressure sensor 400.

When it is determined that a touch is made, the display device 10 operates in a blood pressure measurement mode. For example, when the user sets the blood pressure measurement mode through a program or application installed in the display device 10 before measuring the blood pressure, the display device 10 measures blood pressure when a touch is made. Alternatively, the display device 10 can automatically switch to the blood pressure measurement mode as soon as a touch is made, without the user's intervention. If a user touches a position that is not the blood pressure measurement position, the display device 10 operates in a touch mode. If the user touches a position associated with the blood pressure measurement position, the display device 10 operates in the blood pressure measurement mode. In addition, when the user increases the touch pressure, the pressure sensor 400 operates in the blood pressure measurement mode through pressure analysis. The display device 10 measures blood pressure using both the light-receiving sensor PD and the pressure sensor 400 in the blood pressure measurement mode.

FIG. 6 is a cross-sectional view of the structure of the cover window 100, the display panel 300, the pressure sensor 400, the light-emitting member 740 and the light-receiving sensor PD, taken along line I-I′ of FIG. 4.

As shown in FIG. 6, in an embodiment, some of the light emitted from the light-emitting member 740 through the through hole TH toward the front side of the cover window 100 is reflected by the user's finger OBJ, and is sensed by the light-receiving sensor PD of the display panel 300 through the cover window 100, as shown in step 54 of FIG. 5.

The blood ejected from the left ventricle of a heart moves to the peripheral tissues, and accordingly the blood volume in the artery increases. In addition, red blood cells carry more oxygen in hemoglobin to the peripheral tissues during the systole of the heart. In a diastole of the heart, a part of the blood is sucked from the peripheral tissues towards the heart. When a peripheral blood vessel is irradiated with light, the irradiated light is absorbed by the peripheral tissue. The light absorbance depends on the hematocrit ratio and the blood volume. The light absorbance has a maximum value in the systole of the heart and a minimum value in the diastole of the heart. Therefore, the light sensed by the light-receiving sensor PD is the least during the systole of the heart and the greatest during the diastole of the heart.

In addition, when, in the blood pressure measurement mode, a user touches the display device 10 with her/his finger and then removes the finger from the display device 10, the pressure (contact pressure) applied to the pressure sensor 400 gradually increases to reach the maximum value and then gradually decreases, as shown in step 52 of FIG. 5. As the contact pressure increases, the blood vessels may constrict, resulting in a small or zero blood flow rate. When the contact pressure decreases, the blood vessels dilate and blood begins to flow again. When the contact pressure further decreases, the blood flow rate increases more. Therefore, a change in the amount of light sensed by the light-receiving sensor PD may be proportional to a change in blood flow. The pressure is measured at step 53 of FIG. 5.

FIG. 7 is a graph for calculating blood pressure by a main processor according to an embodiment of the present disclosure.

Referring to FIG. 7, in an embodiment, the main processor 710 generates a pulse wave signal, shown at step 55 of FIG. 5, according to the pressure applied by the user based on the pressure value, shown in the pressure sensor ADC curve, calculated by the pressure sensor 400 and the optical signal, shown in the PPG Signal Ratio curve, according to the amount of light sensed by the light-receiving sensor PD, and calculates a blood pressure at step 56 of FIG. 5 based on the pulse wave signal. The pulse wave signal has a waveform that oscillates according to a heartbeat cycle. For example, the main processor 710 estimates blood pressures of the blood vessels of the user's finger OB3 based on time differences between time points PKT in the pressure sensor ADC curve that correspond to the peaks PK of the calculated pulse wave signal and time points that correspond to the peaks of the filtered pulse wave signal.

The main processor 710 calculates pulse wave signals for predetermined time periods T1 and T2 before and after the time points PKT that correspond to the peaks PK of the calculated pulse wave signal and detects the blood pressure according to differences between the pulse wave signals. Among the estimated blood pressures, a maximum blood pressure is calculated as the systolic blood pressure, and a minimum blood pressure is calculated as the diastolic blood pressure. In addition, other blood pressures, such as the average blood pressure, can be calculated using the estimated blood pressures. The calculated blood pressure is displayed to the user through the display area DA of the display device 10.

The above-described method of measuring blood pressure is merely illustrative, and other methods are known in the art.

Although the user's finger OBJ has been described above as a part of the user's body for which blood pressure is measured in FIGS. 4 to 6, embodiments of the present disclosure are not necessarily limited thereto. For example, the part of the user's body from which blood pressure is measured may be a wrist or another body part where blood vessels are present.

FIG. 6 shows a cross section of the display device 10, taken along line I-I′ of FIG. 4. The bottom cover 900 is not depicted in FIG. 6 for convenience of illustration. In an example shown in FIG. 6, the display device 10 further includes a panel bottom cover 800 on the rear side of the pressure sensor 400.

Referring to FIG. 6, in an embodiment, the display device 10 includes the cover window 100, the display panel 300, the light-receiving sensor PD of the display panel 300, the pressure sensor 400, the bracket 600, the light-emitting member 740, the main circuit board 700, and the panel bottom cover 800.

The pressure sensor 400 is disposed on one surface of the display panel 300. For example, the pressure sensor 400 is disposed on the rear surface (or lower surface) in the third direction (z-axis direction) of the display panel 300. The upper surface of the pressure sensor 400 is attached to the rear surface of the display panel 300 by a transparent adhesive member.

The pressure sensor 400 overlaps the display area DA of the display panel 300 in the third direction (z-axis direction). In an embodiment, the pressure sensor 400 completely overlaps the display area DA of the display panel 300 in the third direction (z-axis direction). Alternatively, in an embodiment, a part of the pressure sensor 400 overlaps the display area DA of the display panel 300 in the third direction DR3 (z-axis direction) and the other part of the pressure sensor 400 overlaps the non-display area NDA of the display panel 300 in the third direction DR3 (z-axis direction).

The pressure sensor 400 includes a first optical hole LH1. In an embodiment, the first optical hole LH1 is an optical hole through which light can pass. Alternatively, in an embodiment, the first optical hole LH1 is physically formed and penetrates through the pressure sensor 400. Alternatively, in an embodiment, the first optical hole L H is a combination of a physical hole and an optical hole.

The through hole TH of the display panel 300 completely overlaps the first optical hole LH1 of the pressure sensor 400. The through hole TH1 of the display panel 300 is smaller than the first optical hole LH1 of the pressure sensor 400. A width of the through hole TH is less than a width of the first optical hole LH1. For example, as shown in FIG. 6, the width of the through hole TH in the first direction (x-axis direction) is less than the width of the first optical hole LH1 in the first direction (x-axis direction).

In an embodiment, a polarizing film is disposed between the display panel 300 and the cover window 100. The polarizing film includes a first base member, a linear polarizer, a λ/4 (quarter-wave) plate, a λ/2 (half-wave) plate, or a second base member. The first base member, the λ/4 plate, the λ/2 plate, the linear polarizer and the second base member are sequentially stacked on the display panel 300.

The panel bottom cover 800 is attached to the lower surface of the pressure sensor 400 by an adhesive member. In an embodiment, the adhesive member is a pressure-sensitive adhesive (PSA). For example, the panel bottom cover 800 includes at least one of a light-blocking member that absorbs externally incident light, a buffer member that absorbs an external impact, or a heat dissipating member that discharges heat from the display panel 300.

The panel bottom cover 800 includes a second optical hole LH2. In an embodiment, the second optical hole LH2 is an optical hole through which light can pass. Alternatively, in an embodiment, the second optical hole LH2 is physically formed and penetrates through the panel bottom cover 800. Alternatively, in an embodiment, the second optical hole LH2 is a combination of a physical hole and an optical hole.

The through hole TH of the display panel 300 completely overlaps the second optical hole LH2 of the panel bottom cover 800. The through hole TH of the display panel 300 is smaller than the second optical hole LH2 of the panel bottom cover 800. A width of the through hole TH is less than a width of the second optical hole 112. For example, as shown in FIG. 6, the width of the through hole TH in the first direction (x-axis direction) is less than the width of the second optical hole LH2 in the first direction (x-axis direction).

In addition, the first optical hole LH1 of the pressure sensor 400 completely overlaps the second optical hole LH2 of the panel bottom cover 800. The first optical hole LH1 of the pressure sensor 400 is smaller than the second optical hole LH2 of the panel bottom cover 800. A width of the first optical hole LH11 is less than a width of the second optical hole LH2. For example, as shown in FIG. 6, the width of the first optical hole LH1 in the first direction (x-axis direction) is less than the width of the second optical hole LH2 in the first direction (x-axis direction).

The bracket 600 is disposed on one surface of the pressure sensor 400. In an embodiment, the bracket 600 is disposed on the rear surface (or lower surface) in the third direction (z-axis direction) of the pressure sensor 400. The bracket 600 includes the sensor hole SH, which is a physical hole that penetrates through the bracket 600. Alternatively, in an embodiment, the sensor hole SH is an optical hole through which light can pass. Alternatively, in an embodiment, the sensor hole SH is a combination of a physical hole and an optical hole.

The through hole TH of the display panel 300 completely overlaps the sensor hole SH of the bracket 600. The through hole TH of the display panel 300 is smaller than the sensor hole SH of the bracket 600. The width of the through hole TH is less than the width of the sensor hole SH. For example, as shown in FIG. 6, the width of the through hole TH in the first direction (x-axis direction) is less than the width of the sensor hole SH in the first direction (x-axis direction).

In addition, both the first optical hole LH1 of the pressure sensor 400 and the second optical hole LH2 completely overlap the sensor hole SH of the bracket 600. The first optical hole LH1 of the pressure sensor 400 is smaller than the sensor hole SH of the bracket 600. The width of the first optical hole LH1 is less than the width of the sensor hole SH. For example, as shown in FIG. 6, the width of the first optical hole LH1 in the first direction (x-axis direction) is less than the width of the sensor hole SH in the first direction (x-axis direction). The size of the second optical hole LH2 is substantially the same as the size of the sensor hole SH. The width of the second optical hole LH1 is substantially equal to the width of the sensor hole SH. For example, as shown in FIG. 6, the width of the second optical hole LH2 in the first direction (x-axis direction) is substantially equal to the width of the sensor hole SH in the first direction (x-axis direction). Therefore, light emitted from the light-emitting member 740 is absorbed or reflected from the blood vessel of the user's finger OBJ through the sensor hole SH, the second optical hole LH2, the first optical hole LH1 and the through hole TH. In addition, light reflected from the blood vessel of the user's finger OBJ is sensed by at least one light-receiving sensor PD disposed or formed adjacent to the through hole TH of the display panel 300.

The wavelength of light emitted by the light-emitting member 740 is one or more of an infrared wavelength or a visible light wavelength, such as red light or green light. When the body part placed on the through hole TH is the finger OBJ, which includes fine blood vessels, infrared and red light emitted by the light-emitting member 740 easily penetrate into and are absorbed by the blood vessels of the finger because they have longer wavelengths than green light or blue light. When the body part placed on the through hole TH is a wrist, which has a thick artery, even the green light emitted by the light-emitting member 740 can penetrate into the wrist artery and be absorbed by it. As such, the wavelength of the light emitted by the light-emitting member 740 can be determined based on which body part the blood pressure is measured from.

The light-emitting member 740 is disposed on the main circuit board 700, and is disposed on a surface of the main processor 710. In an embodiment, the light-emitting member 740 may be mounted on the front surface of the main circuit board 700 or on the upper surface of the main processor 710.

The light-emitting member 740 is disposed at a location that overlaps the through hole TH in the third direction (z-axis direction). Accordingly, the light-emitting member 740 is disposed in the sensor hole SH of the bracket 600. Alternatively, in an embodiment, when the light-emitting emitting member 740 is long in the third direction (z-axis direction), the light-emitting member 740 is disposed in the second optical hole LH2 of the panel bottom cover 800 and the first optical hole LH1 of the pressure sensor 400 or in all the through hole TH of the display panel 300, the second optical hole LH2 of the panel bottom cover 800 and the first optical hole LH1 of the pressure sensor 400. In an embodiment, all of the through hole TH, the first optical hole LH1 and the second optical hole LH2 are physical holes.

As shown in FIG. 6, the light emitted from the light-emitting member 740 is absorbed by or reflected from the blood vessel of the user's finger OBJ through the second optical hole LH2 of the panel bottom cover 800, the first optical hole LH1 of the pressure sensor 400 and the through hole TH of the display panel 300. In addition, the light reflected from the blood vessel of the user's finger OBJ is sensed by at least one light-receiving sensor PD adjacent to the through hole TH of the display panel 300.

FIG. 8 illustrates a layout of a display area and a through hole of a display panel according to an embodiment.

Referring to FIG. 8, in an embodiment, the display area DA includes a through hole TH, a dead space area DSA, a line area LSA, and a pixel area PXA.

The dead space area DSA surrounds the through hole TH. Pixels PX, scan lines and data lines DL are not disposed in the dead space area DSA. At least one light-receiving sensor PD is disposed in the dead space area DSA.

The line area LSA in which the scan lines and the data lines DL are disposed surrounds the dead space area DSA. Since the pixels PX are not disposed in the line area LSA, the line area LSA is a non-display area that does not display an image. At least one light-receiving sensor PD is disposed in the line area LSA adjacent to the through hole TH.

The scan lines and data lines DL that extend around the through hole TH are disposed in the line area LSA. The scan lines include first initialization scan lines GIp to GI(p+4), write scan lines GWp to GW(p+4), and second initialization scan lines GBp to GB(p+4).

The first initialization scan lines Gp to GI(p+4), the write scan lines GWp to GW(p+4), and the second initialization scan lines GBp to GB(p+4) extend in the first direction (x-axis direction). The first initialization scan lines GIp to GI(p+4), the write scan lines GWp to GW(p+4), and the second initialization scan lines GBp to GB(p+4) are bent in the second direction (y-axis direction). In an embodiment, of the first initialization scan lines GIp to GI(p+4), the write scan lines GWp to GW(p+4) and the second initialization scan lines GBp to GB(p+4), the lines that extend around the through hole TH on the upper side are bent toward the upper side, and the lines that extend around the through hole TH on the lower side are bent toward the lower side. Alternatively, in an embodiment, the first initialization scan lines GIp to GI(p+4), the write scan lines GWp to GW(p+4) and the second initialization scan lines GBp to GB(p+4) are bent in a stair pattern to extend around the through hole TH.

The data lines DL extend in the second direction (y-axis direction). The data lines DL are bent in the first direction (x-axis direction) to extend around the through hole TH. In an embodiment, of the data lines DL, the lines that extend around the through hole TH on the left side are bent toward the left side, and the lines that extend around the through hole TH on the right side are bent toward the right side. Alternatively, in an embodiment, the data lines DL are bent in a stair pattern to extend around the through hole TH.

To reduce the size of the line area LSA, the distance between adjacent scan lines in the line area LSA is less than that in the pixel area PXA. In addition, the distance between adjacent data lines DL in the line area ISA is less than that in the pixel area PXA. In addition, in the line area LSA, the scan lines overlap the data lines DL in the third direction (z-axis direction).

Each of the pixels PX overlaps one of the first initialization scan lines GIp to GI(p+4), one of the write scan lines GWp to GW(p+4), one of the second initialization scan lines GBp to GB(p+4), and one of the data lines DL.

As shown in FIG. 8, the scan lines and the data lines DL extend around the through hole TH in the line area LSA, and the pixels PX are not disposed in the line area LSA. Accordingly, even though the through hole TH penetrates through the display area DA of the display panel 300, the display panel 300 can display an image.

FIG. 9 is a cross-sectional view of a display panel of FIG. 8. FIG. 9 is a cross section of the display panel 300 taken along line II-II′ of FIG. 8.

Referring to FIG. 9, in an embodiment, a first buffer layer BF1 is disposed on a substrate SUB, and a thin-film transistor layer TFTL, an emission material layer EML, an encapsulation layer TFE, and a touch electrode layer SENL are sequentially disposed on the first buffer layer BF1.

In an embodiment, at least one light-receiving sensor PD is disposed on the first buffer layer BF1 in the dead space area DSA. Alternatively, in an embodiment, at least one light-receiving sensor PD is disposed on the first buffer layer BF1 in the line area LSA. In the following, an embodiment is described in which at least one light-receiving sensor PD is disposed in the dead space area DSA, but embodiments of the present disclosure are not necessarily limited thereto.

The substrate SUB is made of an insulating material such as glass, quartz and a polymer resin. In an embodiment, the substrate SUB includes polyimide. The substrate SUB is a flexible substrate that can be bent, folded, or rolled.

The first buffer layer BF1 protects the thin-film transistors TFT of the thin-film transistor layer TFTL and an emissive layer 172 of the emission material layer EML. The first buffer layer BF1 includes multiple inorganic layers that are alternately stacked on one another. In an embodiment, the first buffer layer BF1 includes multiple layers in which one or more inorganic layers of a silicon nitride layer, a silicon oxynitride layer, a silicon oxide layer, a titanium oxide layer or an aluminum oxide layer are alternately stacked on one another.

The thin-film transistor layer TFTL in the pixel area PXA includes an active layer ACT, a source electrode S, a drain electrode D, a gate insulator 130, a first interlayer dielectric layer 141, a second interlayer dielectric layer 142, a first planarization layer 160 and a second planarization layer 180.

The light-receiving sensor PD is formed on the first buffer layer BF1 in the dead space area DSA or the line area LSA. When the light-receiving sensor PD is implemented as photo diode (or photo transistor), etc., the light-receiving sensor PD includes a first electrode P1, the gate insulator 130, and a second electrode P2. The first electrode P1 of the light-receiving sensor PD is formed in the same manner as the active layer ACT and performs the same function as the active layer ACT. The second electrode P2 allows a current cap to be formed on the gate insulator 130 according to the amount of the received light, and the amount of current of the first electrode P1 to vary as well. In addition, the first interlayer dielectric layer 141, the second interlayer dielectric layer 142, the first planarization layer 160 and the second planarization layer 180 are further formed on the light-receiving sensor PD. When the light-receiving sensor PD is implemented as a CMOS image sensor or a CCD sensor, the light-receiving sensor PD is disposed on one of: the first buffer layer BF1, the first interlayer dielectric layer 141, the second interlayer dielectric layer 142, the first planarization layer 160 or the second planarization layer 180.

The first electrode P1 and the active layer ACT include one of polycrystalline silicon, single crystal silicon, low-temperature polycrystalline silicon, amorphous silicon, or an oxide semiconductor. When the first electrode P1 and the active layer ACT are made of polycrystalline, active layer ACT is ion-doped to be conductive. Therefore, a partial surface of the first electrode P1, the source electrode S and the drain electrode D is formed by doping the active layer ACT with ions.

The gate insulator 130 is disposed on the first buffer layer BF1, the first electrode P1, the active layer ACT, the source electrode S and the drain electrode D. The gate insulator 130 is formed of an inorganic layer, such as a silicon nitride layer, a silicon oxynitride layer, a silicon oxide layer, a titanium oxide layer, or an aluminum oxide layer.

A gate electrode G, a second electrode P2 and a first capacitor electrode CE1 is formed on the gate insulator 130. The gate electrode G, the second electrode P2 and the first capacitor electrode CE1 may be a single layer or include multiple layers of one of molybdenum (Mo), aluminum (Al), chromium (Cr), gold (Au), titanium (Ti), nickel (Ni), neodymium (Nd) or copper (Cu), or an alloy thereof.

The first interlayer dielectric layer 141 is disposed on the gate insulator 130, the gate electrode G and the first capacitor electrode CE1. The first interlayer dielectric layer 141 is formed of an inorganic layer, such as a silicon nitride layer, a silicon oxynitride layer, a silicon oxide layer, a titanium oxide layer, or an aluminum oxide layer. The first interlayer dielectric layer 141 may include a number of inorganic layers.

A second capacitor electrode CE2 is disposed on the first interlayer dielectric layer 141. The second capacitor electrode CE2 may be a single layer or include multiple layers of one of molybdenum (Mo), aluminum (Al), chromium (Cr), gold (Au), titanium (Ti), nickel (Ni), neodymium (Nd) or copper (Cu), or an alloy thereof.

The second interlayer dielectric layer 142 may be disposed over the first interlayer dielectric layer 141 and the second capacitor electrode CE2. The second interlayer dielectric layer 142 is formed of an inorganic layer, such as a silicon nitride layer, a silicon oxynitride layer, a silicon oxide layer, a titanium oxide layer, or an aluminum oxide layer. The second interlayer dielectric layer 142 may include a number of inorganic layers.

A first anode connection electrode ANDE1 and data lines DL are disposed on the second interlayer dielectric layer 142. The first anode connection electrode ANDE1 is connected to the source electrode S through a contact hole that penetrates through the gate insulator 130, the first interlayer dielectric layer 141 and the second interlayer dielectric layer 142. The first anode connection electrode ANDE1 and the data lines DL

may be a single layer or include multiple layers of one of molybdenum (Mo), aluminum (Al), chromium (Cr), gold (Au), titanium (Ti), nickel (Ni), neodymium (Nd) or copper (Cu), or an alloy thereof.

The first planarization layer 160 is formed on the second interlayer dielectric layer 142 and the first anode connection electrode ANDE1 to provide a flat surface over the active layer ACT, the source electrode S, the drain electrode D, the gate electrode G, the first capacitor electrode CE1, the second capacitor electrode CE2 and the first anode connection electrode ANDE1. The first planarization layer 160 is formed of an organic layer such as an acryl resin, an epoxy resin, a phenolic resin, a polyamide resin or a polyimide resin. A protective layer is first formed between the first anode connection electrode ANDE1 and the first planarization layer 160. The protective layer is formed of an inorganic layer, such as a silicon nitride layer, a silicon oxynitride layer, a silicon oxide layer, a titanium oxide layer, or an aluminum oxide layer.

A second anode connection electrode ANDE2 is formed on the first planarization layer 160. The second anode connection electrode ANDE2 is connected to the first anode connection electrode ANDE1 through a contact hole that penetrates the first planarization layer 160. The second anode connection electrode ANDE2 may be a single layer or include multiple layers of one of molybdenum (Mo), aluminum (Al), chromium (Cr), gold (Au), titanium (Ti), nickel (Ni), neodymium (Nd) or copper (Cu), or an alloy thereof.

A second planarization layer 180 is formed on the first planarization layer 160 and the second anode connection electrode ANDE2. The second planarization layer 180 is formed from an organic layer such as an acryl resin, an epoxy resin, a phenolic resin, a polyamide resin or a polyimide resin.

FIG. 9 shows the thin-film transistor TFT as being implemented as a top-gate transistor in which the gate electrode G is located above the active layer ACT. However, embodiments of the present disclosure are not necessarily limited thereto. In an embodiment, the thin-film transistor TFT is implemented as a bottom-gate transistor in which the gate electrode G is located below the active layer ACT, or as a double-gate transistor in which the gate electrodes G are disposed above and below the active layer ACT.

The emission material layer EML is formed on the thin-film transistor layer TFTL. The emission material layer EML includes light-emitting elements 170 and a bank 190.

The light-emitting elements 170 and the bank 190 are formed on the first planarization layer 160. Each of the light-emitting elements 170 includes a first light-emitting electrode 171, the emissive layer 172, and a second light-emitting electrode 173.

The first light-emitting electrode 171 is formed on the second planarization layer 180. The first light-emitting electrode 171 is connected to the second anode connection electrode ANDE2 through a contact hole that penetrates through the second planarization layer 180.

Ina top-emission organic light-emitting diode in which light exits from the emissive layer 172 toward the second light-emitting electrode 173, the first light-emitting electrode 171 is made of a highly reflective metal such as a stack structure of aluminum and titanium (Ti/Al/Ti), a stack structure of aluminum and ITO (ITO/Al/ITO), an APC alloy, and/or a stack structure of an APC alloy and ITO (ITO/APC/ITO). The APC alloy is an alloy of silver (Ag), palladium (Pd) and copper (Cu).

The bank 190 separates the first light-emitting electrodes 171 on the second planarization layer 180 and defines an emission area EMA. The bank 190 covers an edge of the first light-emitting electrode 171. The bank 190 is formed of an organic layer such as an acryl resin, an epoxy resin, a phenolic resin, a polyamide resin or a polyimide resin.

In the emission area EMA, the first light-emitting electrode 171, the emissive layer 172 and the second light-emitting electrode 173 are sequentially stacked on each other, so that holes from the first light-emitting electrode 171 and electrons from the second light-emitting electrode 173 can combine with each other in the emissive layer 172 to emit light.

The emissive layer 172 is formed on the first light-emitting electrode 171 and the bank 190. The emissive layer 172 includes an organic material that emits light of a predetermined color. The emissive layer 172 includes a hole transporting layer, an organic material layer, and an electron transporting layer.

The second light-emitting electrode 173 is formed on the emissive layer 172 and the bank 190. The second light-emitting electrode 173 covers the emissive layer 172. The second light-emitting electrode 173 is a common layer formed across the sub-pixels SP1, SP2 and SP3. In an embodiment, a capping layer is formed on the second light-emitting electrode 173.

In a top-emission organic light-emitting diode, the second light-emitting electrode 173 is formed of a transparent conductive material (TCP) such as ITO and IZO that can transmit light, or a semi-transmissive conductive material such as magnesium (Mg), silver (Ag) and/or an alloy of magnesium (Mg) and silver (Ag). When the second light-emitting electrode 173 is formed of a semi-transmissive conductive material, the light extraction efficiency can be increased by using microcavities.

The encapsulation layer TFE is formed on the light-emitting element layer EML. The encapsulation layer TFE includes at least one inorganic layer that prevents permeation of oxygen or moisture into the emission material layer EML and the light-receiving sensor PD. In addition, the encapsulation layer TFE includes at least one organic layer that protects the light-receiving sensor PD and the emission material layer EML from particles such as dust. In an embodiment, the encapsulation layer TFE includes a first inorganic layer TFE1, an organic layer TFE2 and a second inorganic layer TFE3.

The first inorganic layer TFE1 is disposed on the second light-emitting electrode 173, the organic layer TFE2 is disposed on the first inorganic layer TFE1, and the second inorganic layer TFE3 is disposed on the organic layer TFE2. The first inorganic layer TFE) and the second inorganic layer TFE3 include multiple layers in which one or more inorganic layers of a silicon nitride layer, a silicon oxynitride layer, a silicon oxide layer, a titanium oxide layer or an aluminum oxide layer are alternately stacked on one another. The organic layer TFE2 is a monomer.

The touch electrode layer SENL is disposed on the encapsulation layer TFEL. The touch electrode layer SENL includes a second buffer layer BF2, touch electrodes SE, a first touch insulating layer TINS1 and a second touch insulating layer TINS2.

The second buffer layer BF2 is disposed on the encapsulation layer TFEL and the light-receiving sensor PD. The second buffer layer BF2 includes at least one inorganic layer. In an embodiment, the second buffer layer BF2 includes multiple layers in which one or more inorganic layers of a silicon nitride layer, a silicon oxynitride layer, a silicon oxide layer, a titanium oxide layer and an aluminum oxide layer are alternately stacked on each other. In an embodiment, the second buffer layer BF2 is omitted.

The first touch insulating layer TINS1 is disposed on the second buffer layer BF2. The first touch insulating layer TINS1 is formed of an inorganic layer, such as a silicon nitride layer, a silicon oxynitride layer, a silicon oxide layer, a titanium oxide layer, or an aluminum oxide layer. Alternatively, in an embodiment, the first touch insulating layer TINS1 is formed of an organic layer, such as an acryl resin, an epoxy resin, a phenolic resin, a polyamide resin or a polyimide resin.

The touch electrodes SE are disposed on the first touch insulating layer TINS1. The touch electrodes SE do not overlap the emission area EMA. For example, the touch electrodes SE are not disposed in the emission area EMA. The touch electrodes SE may be a single layer of molybdenum (Mo), titanium (Ti), copper (Cu) or aluminum (Al), or may be a stack structure of aluminum and titanium (Ti/Al/Ti), a stack structure of aluminum and ITO (ITO/Al/ITO), an APC alloy, and/or a stack structure of an APC alloy and ITO (ITO/APC/ITO).

The second touch insulating layer TINS2 is disposed on the touch electrodes SE and the first touch insulating layer TINS1. The second touch insulating layer TINS2 includes at least one of an inorganic layer or an organic layer. The inorganic layer may be one of a silicon nitride layer, a silicon oxynitride layer, a silicon oxide layer, a titanium oxide layer, or an aluminum oxide layer. The organic layer may be one of an acryl resin, an epoxy resin, a phenolic resin, a polyamide resin or a polyimide resin.

The cover window 100 is disposed on the touch electrode layer SENL. In an embodiment, a polarizing film and a shock absorbing layer are further disposed between the touch electrode layer SENL and the cover window 100.

A dam structure DAM is disposed around the through hole Till. The dam structure DAM includes at least one of the insulating layers BF1, 130, 141, 142, 160, 180 or 190 stacked on the thin-film transistor layer TFTL and the emission material layer EM. A trench TCH is formed between the dam structure DAM and the emission area EMA by removing a part of the insulating layers BF1, 130, 141, 142, 160 180 and 190. At least a part of the encapsulation layer TFE is disposed in the trench TCH. For example, the organic layer TFE2 of the encapsulation layer TFE is disposed in the trench and up to the dam structure DAM, but is not disposed between the dam structure DAM and the through hole TH. The dam structure DAM prevents a first organic layer 228 from overflowing into the through hole TH. Although the first inorganic layer TFE1 and the second inorganic layer TFE3 terminate on the dam structure DAM in the example shown in FIG. 9, embodiments of the present disclosure are not necessarily limited thereto. In an embodiment, the first inorganic layer TFE1 and the second inorganic layer TFE3 terminate between the dam structure DAM and the through hole TH.

The first organic layer 228 and a second organic layer 229 are further disposed on the light-receiving sensor PD between the dam structure DAM and the through hole TH. For example, the first organic layer 228 is disposed on the second inorganic layer TFE3, and the second organic layer 229 is disposed on the first organic layer 228. The space between the dam structure DAM and the through hole TH is filled with the first organic layer 228 and the second organic layer 229, to provide a flat surface.

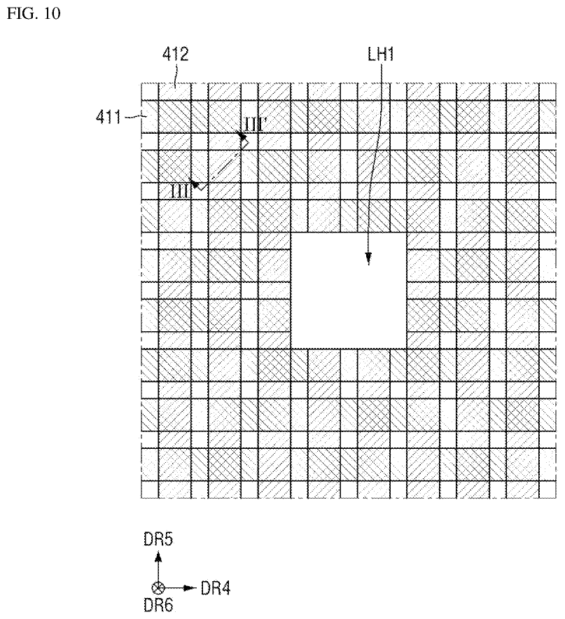

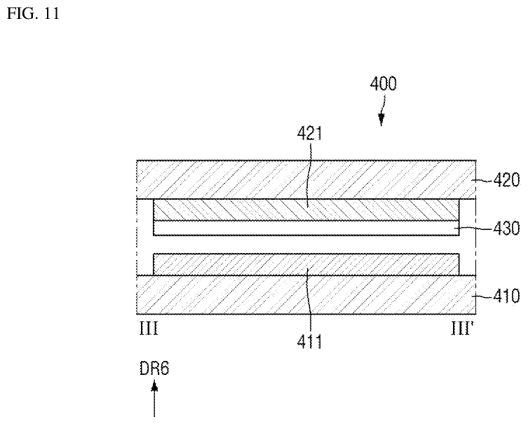

FIG. 10 illustrates a layout of pressure sensor electrodes of a pressure sensor and a first optical hole according to an embodiment of the present disclosure. FIG. 11 is a cross-sectional of a pressure sensor of FIG. 10. FIG. 11 is a cross section of the pressure sensor 400, taken along III-III′ of FIG. 10.

Referring to FIGS. 10 to 11, in an embodiment, the pressure sensor 400 includes a first base substrate 410, a first pressure sensor electrode 411, a second base substrate 420, a second pressure sensor electrode 421, and a pressure sensing layer 430 disposed between the first pressure sensor electrode 411 and the second pressure sensor electrode 421.

Each of the first base substrate 410 and the second base substrate 420 includes a material such as polyethylene, polyimide, polycarbonate, polysulfone, polyacrylate, polystyrene, polyvinyl chloride, polyvinyl alcohol, poly(norbornene), or polyester. According to an embodiment of the present disclosure, each of the first base substrate 410 and the second base substrate 420 is a polyethylene terephthalate (PET) film or a polyimide film.

The first base substrate 410 and the second base substrate 420 are coupled to each other by a coupling layer. The coupling layer includes an adhesive material. The coupling layer is disposed along the edges of the first base substrate 410 and the second base substrate 420, but embodiments the present disclosure are not necessarily limited thereto.

The first pressure sensor electrodes 411 are disposed on a surface of the first base substrate 410 that faces the second base substrate 420. The second pressure sensor electrodes 421 are disposed on a surface of the second base substrate 420 that faces the first base substrate 410. Each of the first pressure sensor electrodes 411 and the second pressure sensor electrodes 421 includes a conductive material. For example, each of the first pressure sensor electrodes 411 and the second pressure sensor electrodes 421 includes a metal such as silver (Ag) or copper (Cu), a transparent conductive oxide such as ITO, IZO or ZIO, carbon nanotubes, conductive polymers, etc. One of the first pressure sensor electrode 411 and the second pressure sensor electrode 421 may be a pressure driving electrode, while the other may be a pressure sensing electrode.

The pressure sensing layer 430 is disposed between the first pressure sensor electrode 411 and the second pressure sensor electrode 421. The pressure sensing layer 430 is in contact with at least one of the first pressure sensor electrode 411 and the second pressure sensor electrode 421. For example, the pressure sensing layer 430 is in contact with the second pressure sensor electrode 421 as shown in FIG. 11, or may be in contact with the first pressure sensor electrode 411.

The pressure sensing layer 430 contains a pressure sensitive material. The pressure sensitive material includes metal nanoparticles such as nickel, aluminum, tin, copper, or carbon. The pressure-sensitive material is dispersed in a polymer resin in the form of particles. However, embodiments of the present disclosure are not necessarily limited thereto.

When pressure is applied to the pressure sensor 400, the first pressure sensor electrode 411, the pressure sensing layer 430 and the second pressure sensor electrode 421 become electrically connected with each other. The electrical resistance of the pressure sensing layer 430 is lowered according to the pressure applied to the pressure sensor 400. The electrical resistance of the pressure sensing layer 430 is calculated by applying a pressure driving voltage to the first pressure sensor electrode 411 and measuring the pressure sensing voltage through the second pressure sensor electrode 421. It is possible to determine whether a pressure is applied and if so, to calculate the amount of the pressure based on the electrical resistance of the pressure sensing layer 430.

The first pressure sensor electrodes 411 extend in a fourth direction DR4 and are arranged in a fifth direction DR5 that crosses the fourth direction. The second pressure sensor electrodes 421 extend in the fifth direction DR5 and are arranged in the fourth direction DR4. A sixth direction DR6 is a thickness direction of the pressure sensor 400 and is normal to a plane defined by the fourth direction DR4 and the fifth direction DR5. The first pressure sensor electrodes 411 and the second pressure sensor electrodes 421 cross each other. The intersections of the first pressure sensor electrodes 411 and the second pressure sensor electrodes 421 are arranged in a matrix pattern. Each of the intersections of the first pressure sensor electrodes 411 and the second pressure sensor electrodes 421 is a pressure sensing cell for sensing pressure. For example, a pressure can be sensed in each of the intersections of the first pressure sensor electrodes 411 and the second pressure sensor electrodes 421.

When the first pressure sensor electrode 411 and the second pressure sensor electrode 421 include an opaque conductive material, or the pressure sensing layer 430 includes an opaque polymer resin, the pressure sensor 400 is opaque. To prevent light passing through the through hole TH from being blocked by the pressure sensor 400, the pressure sensor 400 includes a first optical hole LH1. The first optical hole LH1 is formed by removing some of the first pressure sensor electrode 411, the second pressure sensor electrode 421 and the pressure sensing layer 430, which contain an opaque material. For example, when the first pressure sensor electrode 411 and the second pressure sensor electrode 421 include an opaque conductive material, the first pressure sensor electrode 411 and the second pressure sensor electrode 421 are removed from the first optical hole LH1. When the pressure sensing layer 430 includes an opaque polymer resin, the pressure sensing layer 430 is removed from the first optical hole LH1. When the first pressure sensor electrode 411 and the second pressure sensor electrode 421 include an opaque conductive material and the pressure sensing layer 430 includes an opaque polymer resin, the first pressure sensor electrode 411, the second pressure sensor electrode 421 and the pressure sensing layer 430 are removed from the first optical hole LH1.

Alternatively, in an embodiment, the first pressure sensor electrode 411, the second pressure sensor electrode 421 and the pressure sensing layer 430 are included in the first base substrate 410 and the second base substrate 420. For example, the first pressure sensor electrode 411 and the pressure sensing layer 430 are included in the first base substrate 410, and the second pressure sensor electrode 421 is included in the second base substrate 420. Alternatively, in an embodiment, the first pressure sensor electrode 411, the second pressure sensor electrode 421 and the pressure sensing layer 430 are included in one of the first base substrate 410 or the second base substrate 420.

FIG. 10 shows eight first pressure sensor electrodes 411 and eight second pressure sensor electrodes 421 for convenience of illustration. However the numbers of the first pressure sensor electrodes 411 and the second pressure sensor electrodes 421 are not necessarily limited thereto. The length of the pressure sensor 400 in the fourth direction DR4 and the length of the pressure sensor 400 in the fifth direction DR5 are approximately 10 mm to 20 mm. The length of the intersection of the first pressure sensor electrode 411 and the second pressure sensor electrode 421 in the fourth direction DR4 and in the fifth direction DR5 is approximately 1.5 mm or greater. The lengths of the first optical hole LH1 in the fourth direction DR4 and in the fifth direction DR5 are approximately 3 mm or greater.

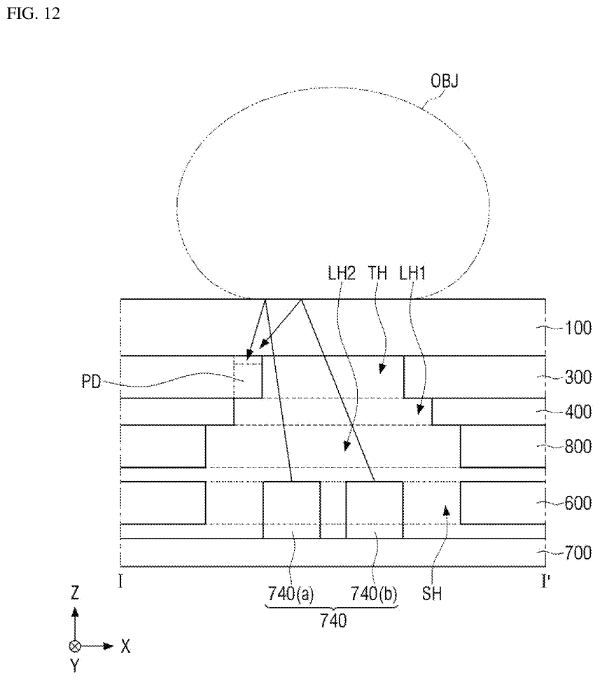

FIG. 12 is another cross-sectional view of the cover window 100, the display panel 300, the pressure sensor 400, the light-emitting member 740 and the light-receiving sensor PD, taken along line I-I′ of FIG. 4. FIG. 12 shows another example of the display device 10, taken along line I-I′ of FIG. 4.

In an example shown in FIG. 12, the bottom cover 900 is not depicted for convenience of illustration, and the panel bottom cover 800 is disposed on the rear side of the pressure sensor 400. An embodiment of FIG. 12 differs from an embodiment of FIG. 6 in that the light-emitting member 740 includes a first light-emitting diode 740(a) and a second light-emitting diode 740(b). Thus, a repeated description of components described with respect to FIG. 6 is omitted.

The light-emitting member 740 includes a first light-emitting diode 740(a) that emits infrared light, and a second light-emitting diode 740(b) that emits green light.

When a body part placed on the through hole TH is a finger OBJ, the infrared light easily penetrates into the blood vessel of the finger and is absorbed. On the other hand, when the body part placed on the through hole TH is a wrist, green or ultraviolet light easily penetrates into the wrist artery and is absorbed. Accordingly, the light-emitting member 740 that includes the first and second light-emitting diodes 740(a) and 740(b) efficiently uses the emission absorption rate and the reflectance when the body part from which blood pressure is measured is a finger or a wrist. In particular, the peak value of the waveform detected in measuring blood pressure can be accurately sensed by the second light-emitting diode 740(b) that emits green light. The wavelength forms that are continuously detected in measuring blood pressure can be accurately analyzed by the first light-emitting diode 740(a) that emits infrared light. As described above, by using light of different wavelength ranges, the absorption rate and reflectance are efficiently used, and further, blood oxygen saturation can be measured based on analysis results of the sensing signal.

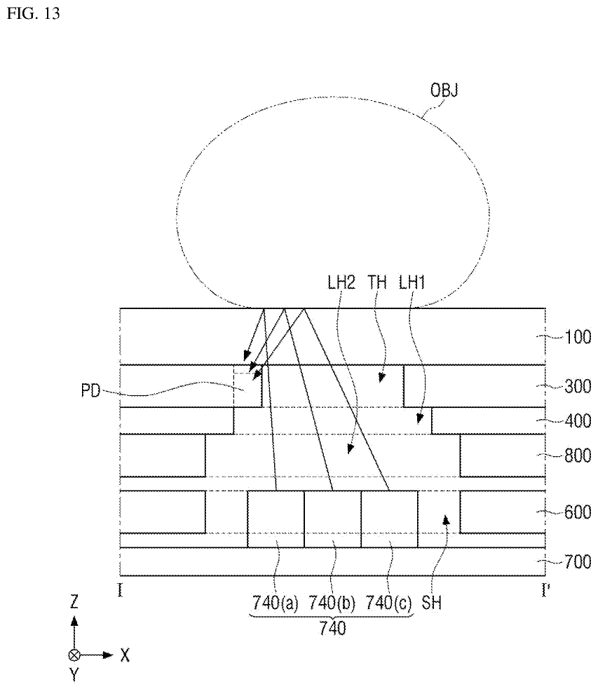

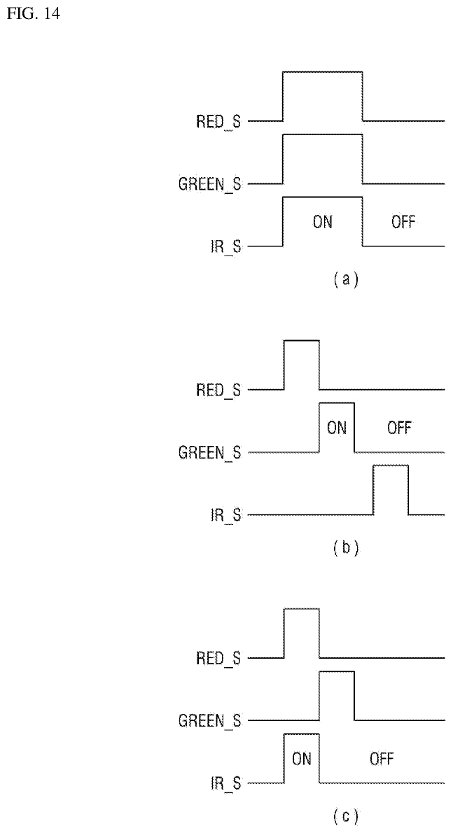

FIG. 13 is another cross-sectional view of a cover window 100, the display panel 300, the pressure sensor 400, the light-emitting member 740 and the light-receiving sensor PD, taken along line I-I′ of FIG. 4. FIG. 14 is a timing diagram that illustrates driving control signals for a light-emitting member shown in FIG. 13.

FIG. 13 shows yet another example of the display device 10, taken along line I-I′ of FIG. 4.

The light-emitting member 740 of FIG. 13 differs from that of FIG. 12 in that the light-emitting member 740 includes a first light-emitting diode 740(a) that emits infrared light, a second light-emitting diode 740(b) that emits green light, and a third light-emitting diode 740(c) that emits visible light of a color other than green light.

Infrared light easily penetrates into blood vessels and is absorbed, and continuously detected waveforms can be accurately analyzed in measuring blood pressure. On the other hand, green light and red light easily penetrate into the artery and are absorbed by it, and the peak value of the waveform can be accurately detected by measuring blood pressure.

As shown in FIG. 13, when infrared light, green light and blue light are combined or sequentially used, a continuously detected waveform that includes the peak value of the waveform can be accurately detected and analyzed by measuring blood pressure.

Referring to FIG. 14(a), the main processor 710 controls the light-emitting member 740 so that the light-emitting diodes 740(a) to 740(c) in the light-emitting member 740 are simultaneously turned on or turned off at a predetermined driving period. For example, the main processor 710 generates first to third driving control signals RED_S, GREEN_S and IR_S to simultaneously turn on or off the first to third light-emitting diodes 740(a) to 740(c) of the light-emitting member 740 at a predetermined driving period. Then, the main processor 710 simultaneously turns on or off the first to third light-emitting diodes 740(a) to 740(c) of the light-emitting member 740 using the first to third driving control signals RED_S, GREEN_S and IR_S, respectively.

Referring to FIG. 14(b), the main processor 710 controls the light-emitting member 740 so that the light-emitting diodes 740(a) to 740(c) in the light-emitting member 740 are sequentially turned on or turned off at different periods. For example, the main processor 710 generates first to third driving control signals RED_S, GREEN_S and IR_S to sequentially turn on or off the first to third light-emitting diodes 740(a) to 740(c) of the light-emitting member 740 at different periods. Then, the main processor 710 sequentially turns on or off the first to third light-emitting diodes 740(a) to 740(c) at different driving periods using the first to third driving control signals RED_S, GREEN_S and IR_S, respectively.

Referring to FIG. 14(c), the main processor 710 controls the light-emitting member 740 so that at least two of the light-emitting diodes 740(a) to 740(c) in the light-emitting member 740, such as the light-emitting diodes 740(a) and 740(c), are simultaneously turned on or turned off at a predetermined first driving period. In addition, the main processor 710 controls the other light-emitting diode 740(b) so that it is sequentially turned on or turned off in a different period from the first driving period.

In an embodiment, the main processor 710 controls the light-emitting member 740 so that the light-emitting diodes 740(a) and 740(c) of the light-emitting member 740 are simultaneously turned on or turned off at a predetermined driving period. On the other hand, main processor 710 controls the other light-emitting diode 740(b) so that it is sequentially turned on or turned off in a different period from that of the first and third light-emitting diodes 740(a) and 740(c).

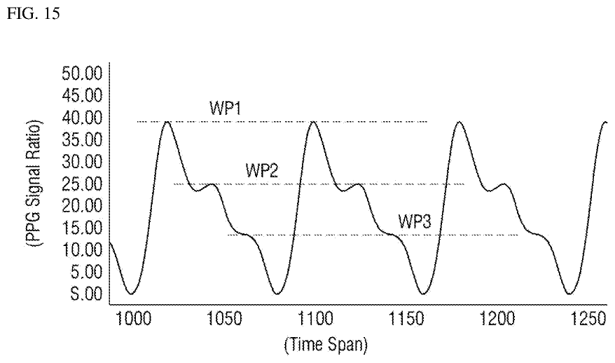

Referring to FIG. 15, in an embodiment, the first to third light-emitting diodes 740(a) to 740(c) may simultaneously or sequentially generate infrared light, green light and blue light, respectively.

The main processor 710 analyzes an optical signal received from the light-receiving sensor PD by dividing the signal into a peak value detection period WP1 according to an infrared light emission period, a first continuous waveform detection period WP2 according to the green light emission period, and a second continuous waveform detection period WP3 according to the blue light emission period. In this manner, the main processor 710 can accurately detect and analyze a peak value of a waveform detected by measuring blood pressure and continuously detecting a waveform.

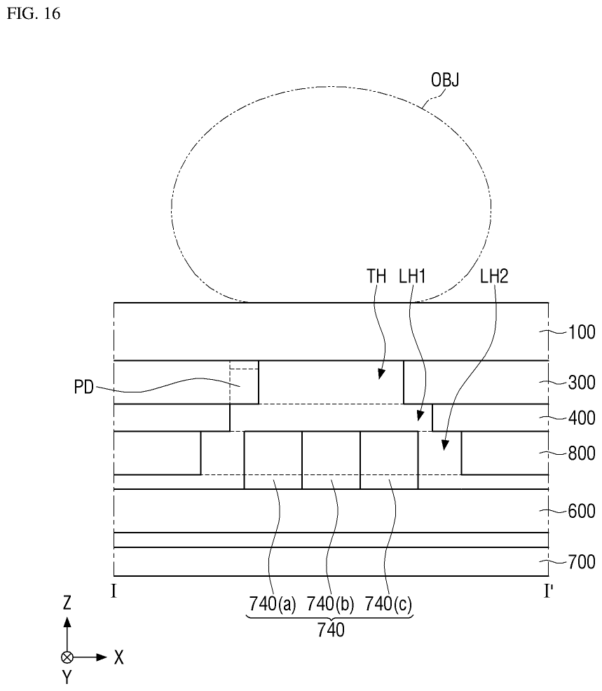

FIG. 16 is a cross-sectional view of the cover window 100, the display panel 300, the pressure sensor 400, the light-emitting member 740 and the light-receiving sensor PD according to an embodiment, taken along line I-I′ of FIG. 4.

Referring to FIG. 16, the first optical hole LH1 of the pressure sensor 400 overlaps the second optical hole 112 of the panel bottom cover 800, and the through hole TH of the display panel 300 overlaps the second optical hole LH2 of the panel bottom cover 800.

The light-emitting member 740 is attached on an upper surface of the bracket 600 and is disposed in the second optical hole LH2 of the panel bottom cover 800.

Therefore, the light emitted from the light-emitting member 740 is absorbed by or reflected from the blood vessel of the user's finger OBJ through the second optical hole LH2, the first optical hole LH1 and the through hole TH. In addition, the light reflected from the blood vessel of the user's finger OB3 is sensed by at least one light-receiving sensor PD disposed or formed in second optical hole LH2 of the panel bottom cover 800.

The light-emitting member 740 is attached on the bracket 600 as shown in FIG. 16, and the light-emitting member 740 is closer to a user's body part than in a structure in which the light-emitting member 740 is attached on the main circuit board 700. As the light-emitting member 740 is closer to a user's body part, the accuracy and efficiency of an optical signal sensed by the at least one light-receiving sensor PD is increased.

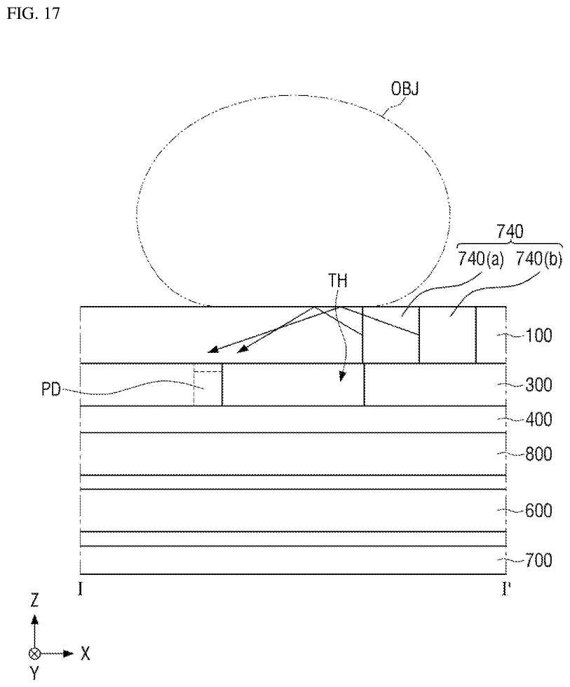

FIG. 17 is a cross-sectional view of the cover window 100, the display panel 300, the pressure sensor 400, the light-emitting member 740 and the light-receiving sensor PD according to an embodiment, taken along line I-I′ of FIG. 4.

Referring to FIG. 17, in an embodiment, the light-emitting member 740 is integrally formed with the cover window 100 or is incorporated into the cover window 100.

When the light-emitting member 740 is integrally formed with the cover window 100 or incorporated into the cover window 100, the light-emitting member 740 is closest to a user's body part. The light emitted from the light-emitting member 740 is absorbed by or reflected from the blood vessels of the user's finger OBJ through the cover window 100. In addition, the light reflected from a user's body part through the cover window 100 is sensed by at least one light-receiving sensor PD formed on the display panel 300.

When the light-emitting member 740 is formed in the cover window 100, the light-emitting member 740 is closer to the user's body part than when the light-emitting member 740 is attached on the main circuit board 700 or the bracket 600. Even though the light reflection path be different, as the light-emitting member 740 is closer to a user's body part, the accuracy and efficiency of an optical signal sensed by the at least one light-receiving sensor PD is increased. When the light-emitting member 740 is formed in the cover window 100, no first optical hole LH1 is formed in the pressure sensor 400, and no second optical hole LH2 is formed in the panel bottom cover 800. For example, the pressure sensor 400, the bracket 600, the panel bottom cover 800, etc. are formed as a flat plate without a hole.

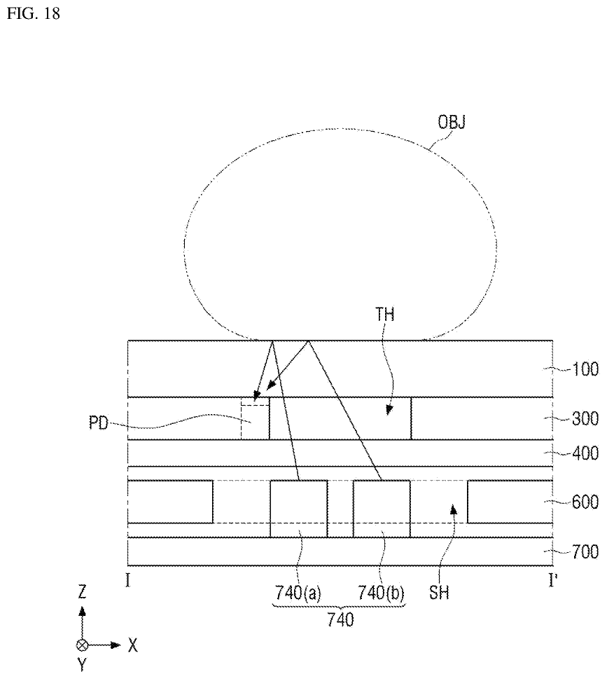

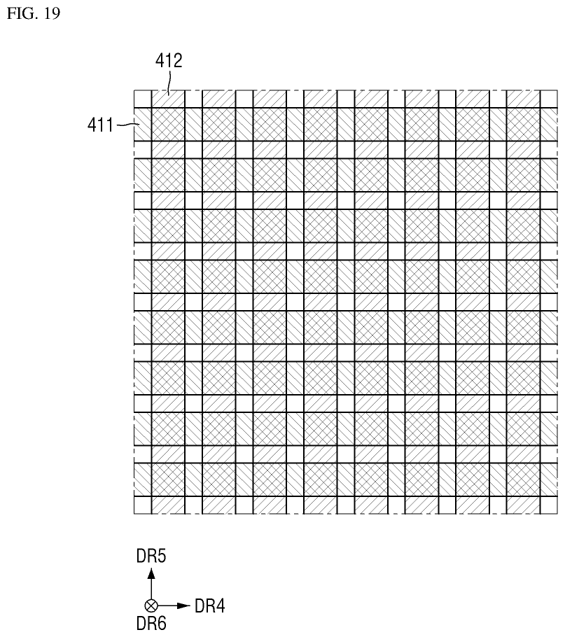

FIG. 18 is a cross-sectional view of the cover window 100, the display panel 300, the pressure sensor 400, the light-emitting member 740 and the light-receiving sensor PD according to an embodiment, taken along line I-I′ of FIG. 4. FIG. 19 illustrates a layout of pressure sensor electrodes of a light-emitting member shown in FIG. 18.

An embodiment of FIGS. 18 and 19 differs from an above-described embodiment in that the first optical hole LH1 and is eliminated from the pressure sensor 400, and there is no panel bottom cover 800.

Referring to FIGS. 18 and 19, in an embodiment, the pressure sensor 400 is transparent so that light can pass therethrough. For example, when the pressure sensor 400 includes the pressure sensing layer 430 (see FIG. 11) to sense pressure, the first base substrate 410 and the second base substrate 420 include a transparent material, the first pressure sensor electrode 411 and the second pressure sensor electrode 421 include a transparent conductive material, and the pressure sensing layer 430 includes a transparent polymer resin. Since the pressure sensor 400 can pass light, the first optical hole LH1 is not required in the pressure sensor 400, as shown in FIGS. 18 and 19.

The light-emitting member 740 is disposed without the panel bottom cover 800. Specifically, the light-emitting member 740 is disposed on the main circuit board 700 with the panel bottom cover 800 removed. As such, the light-emitting member 740 is attached to the main circuit board 700 and disposed in the sensor hole SH of the bracket 600.

Accordingly, the light emitted from the light-emitting member 740 passes through the transparent pressure sensor 400 and is absorbed by or reflected from the blood vessel of the user's finger OBJ through the through hole TH. In addition, the light reflected from the blood vessel of the user's finger OBJ is sensed by at least one light-receiving sensor PD disposed or formed in the through hole TH of the display panel 300.

When the light-emitting member 740 is disposed on the main circuit board 700 without the panel bottom cover 800, the light-emitting member 740 is closer to a user's body part. As the light-emitting member 740 is closer to a user's body part, the accuracy and efficiency of an optical signal sensed by the at least one light-receiving sensor PD is increased.

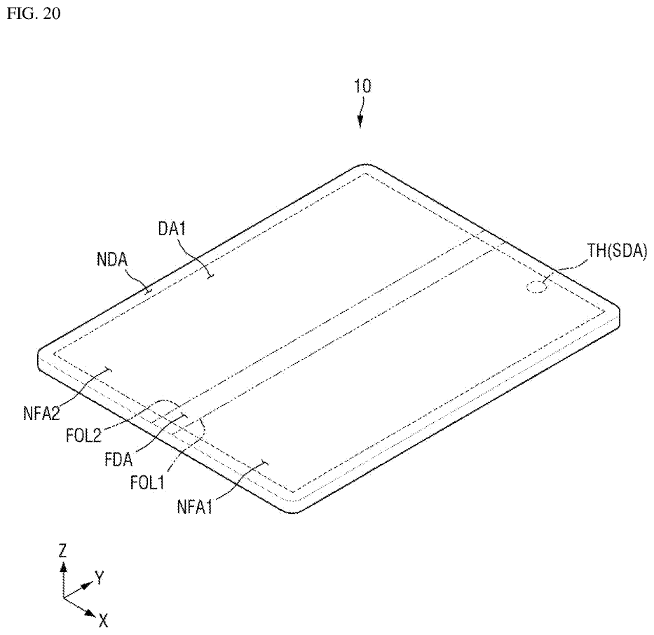

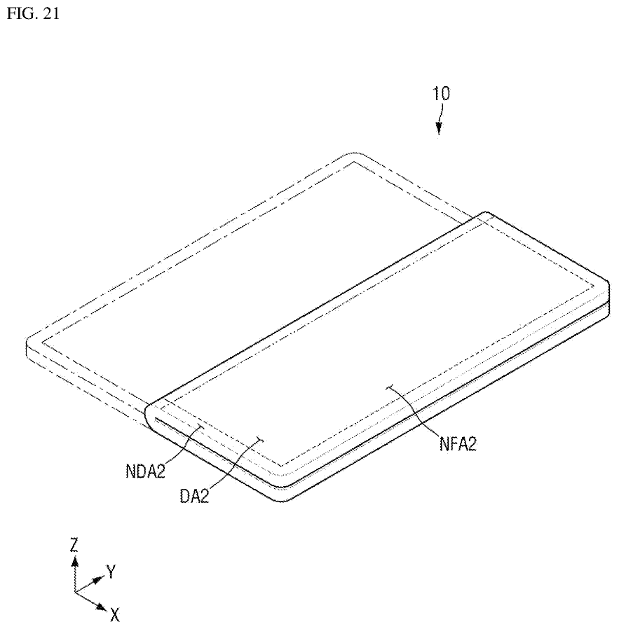

FIGS. 20 and 21 are perspective views of a display device according to an embodiment.

In an embodiment shown in FIGS. 20 and 21, a display device 10 is a foldable display device that is folded in the first direction (x-axis direction). The display device 10 may remain folded as well as unfolded. The display device 10 may be folded inward (in-folding manner) such that the front surface is located inside. When the display device 10 is bent or folded in an in-folding manner, a part of the front surface of the display device 10 faces the other part of the front surface. Alternatively, the display device 10 may be folded outward (out-folding manner) such that the front surface is located outside. When the display device 10 is bent or folded in an out-folding manner, a part of the rear surface of the display device 10 faces the other part of the rear surface.

The display device 10 includes a folding area FDA, a first non-folding area NFA1, and a second non-folding area NFA2. The display device 10 can be folded at the folding area FDA, while it cannot be folded at the first non-folding area NFA1 and the second non-folding area NFA2.

The first non-folding area NFA1 is disposed on one side, for example, the right side of the folding area FDA. The second non-folding area NFA2 is disposed on the other side, for example, the left side of the folding area FDA. The folding area FDA is an area that can be bent with a predetermined curvature over a first folding line FOL1 and a second folding line FOL2. Therefore, the first folding line FOL1 is a boundary between the folding area FDA and the first non-folding area NFA1, and the second folding line FOL2 is a boundary between the folding area FDA and the second non-folding area NFA2.

The first folding line FOL1 and the second folding line FOL2 extend in the second direction (y-axis direction), and the display device 10 can be folded in the first direction (x-axis direction). As a result, the length of the display device 10 in the first direction (the x-axis direction) is reduced to about half, so that a user can carry the display device 10 more easily.

The direction in which the first folding line FOL1 and the second folding line FOL2 extend is not limited to the second direction (y-axis direction). In an embodiment, the first folding line FOL1 and the second folding line FOL2 extend in the first direction (x-axis direction), and the display device 10 is folded in the second direction (y-axis direction). In such case, the length of the display device 10 in the second direction (y-axis direction) is reduced to about half. Alternatively, in an embodiment, the first folding line FOL1 and the second folding line FOL2 extend in a diagonal direction of the display device 10 between the first direction (x-axis direction) and the second direction (y-axis direction). The display device 10 can fold in a triangle shape.

When the first folding line FOL1 and the second folding line FOL2 extend in the second direction (y-axis direction), the length of the folding area FDA in the first direction (x-axis direction) is less than it's length in the second direction (y-axis direction). In addition, the length of the first non-folding area NFA1 in the first direction (x-axis direction) is greater than the length of the folding area FDA in the first direction (x-axis direction). The length of the second non-folding area NFA2 in the first direction (x-axis direction) is greater than the length of the folding area FDA in the first direction (x-axis direction).

A first display area DA1 is disposed on the front side of the display device 10. The first display area DA1 overlaps the folding area FDA, the first non-folding area NFA1, and the second non-folding area NFA2. Therefore, when the display device 10 is unfolded, images can be displayed on the front side of the folding area FDA, the first non-folding area NFA1 and the second non-folding area NFA2 of the display device 10.

The second display area DA2 is disposed on the rear side of the display device 10. The second display area DA2 overlaps the second non-folding area NFA2. Therefore, when the display device 10 is folded, images can be displayed on the front side of the second non-folding area NFA2 of the display device 10.

Although the through hole TH or the subsidiary display area SDA are disposed in the first non-folding area NFA1 in FIGS. 20 and 21, embodiments of the present disclosure are not necessarily limited thereto. In an embodiment, the through hole TH or the subsidiary display area SDA is disposed in the second non-folding area NFA2 or the folding area FDA.

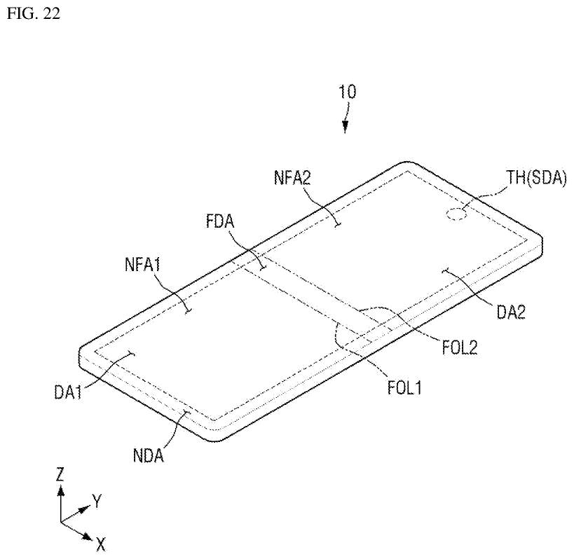

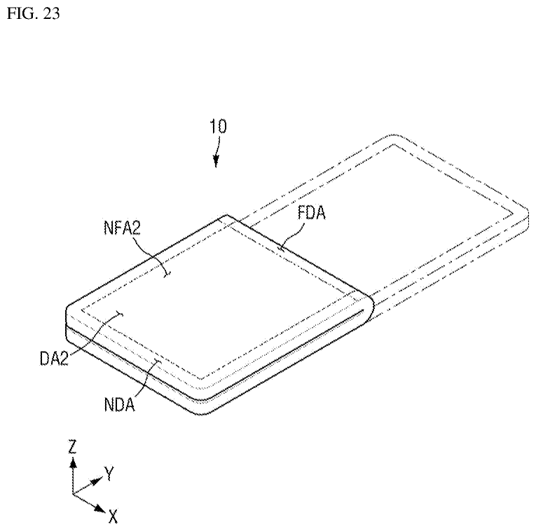

FIGS. 22 and 23 are perspective views of a display device according to an embodiment of the present disclosure.

In an embodiment shown in FIGS. 22 and 23, a display device 10 is a foldable display device that is folded in the second direction (y-axis direction). The display device 10 may remain folded as well as unfolded. The display device 10 may be folded inward (in-folding manner) such that the front surface is located inside. When the display device 10 is bent or folded in the in-folding manner, a part of the front surface of the display device 10 faces the other part of the front surface. Alternatively, the display device 10 may be folded outward (out-folding manner) such that the front surface is located outside. When the display device 10 is bent or folded in the out-folding manner, a part of the rear surface of the display device 10 faces the other part of the rear surface.

The display device 10 includes a folding area FDA, a first non-folding area NFA1, and a second non-folding area NFA2. The display device 10 can be folded at the folding area FDA, while it cannot be folded at the first non-folding area NFA1 and the second non-folding area NFA2.

The first non-folding area NFA1 is disposed on one side, for example, the lower side of the folding area FDA. The second non-folding area NFA2 is disposed on the other side, for example, the upper side of the folding area FDA. The folding area FDA is an area that can be bent with a predetermined curvature over a first folding line FOL1 and a second folding line FOL2. Therefore, the first folding line FOL1 is a boundary between the folding area FDA and the first non-folding area NFA1, and the second folding line FOL2 is a boundary between the folding area FDA and the second non-folding area NFA2.

The first folding line FOL1 and the second folding line FOL2 extend in the first direction (x-axis direction) as shown in FIGS. 22 and 23, and the display device 10 can be folded in the second direction (y-axis direction). As a result, the length of the display device 10 in the second direction (the y-axis direction) is reduced to about half, so that the display device 10 is easier to carry.

The direction in which the first folding line FOL1 and the second folding line FOL2 extend is not limited to the first direction (x-axis direction). In an embodiment, the first folding line FOL1 and the second folding line FOL2 extend in the second direction (y-axis direction), and the display device 10 can be folded in the first direction (x-axis direction). In such case, the length of the display device 10 in the first direction (x-axis direction) is reduced to about half. Alternatively, in an embodiment, the first folding line FOL1 and the second folding line FOL2 extend in a diagonal direction of the display device 10 between the first direction (x-axis direction) and the second direction (y-axis direction). The display device 10 can fold in a triangle shape.

When the first folding line FOL1 and the second folding line FOL2 extend in the first direction (x-axis direction) as shown in FIGS. 22 and 23, the length of the folding area FDA in the second direction (y-axis direction) is less than the length in the first direction (x-axis direction). In addition, the length of the first non-folding area NFA1 in the second direction (y-axis direction) is greater than the length of the folding area FDA in the second direction (y-axis direction). The length of the second non-folding area NFA2 in the second direction (y-axis direction) is greater than the length of the folding area FDA in the second direction (y-axis direction).

A first display area DA1 is disposed on the front side of the display device 10. The first display area DA1 overlaps the folding area FDA, the first non-folding area NFA1, and the second non-folding area NFA2. Therefore, when the display device 10 is unfolded, images can be displayed on the front side of the folding area FDA, the first non-folding area NFA1 and the second non-folding area NFA2 of the display device 10.

A second display area DA2 may be disposed on the rear side of the display device 10. The second display area DA2 overlaps the second non-folding area NFA2. Therefore, when the display device 10 is folded, images can be displayed on the front side of the second non-folding area NFA2 of the display device 10.

Although the through hole TH or the subsidiary display area SDA are disposed in the second non-folding area NFA2 in FIGS. 22 and 23, embodiments of the present disclosure are not necessarily limited thereto. In an embodiment, the through hole TH or the subsidiary display area SDA are disposed in the first non-folding area NFA1 or the folding area FDA.

Although embodiments of the present disclosure have been described with reference to the accompanying drawings, those skilled in the art would understand that various modifications and alterations may be made without departing from the technical idea or essential features of embodiments of the present disclosure. Therefore, it should be understood that above-mentioned embodiments are not limiting but illustrative in all aspects.

In concluding the detailed description, those skilled in the art will appreciate that many variations and modifications can be made to embodiments without substantially departing from the principles of the present disclosure. Therefore, embodiments of the disclosure are used in a generic and descriptive sense only and not for purposes of limitation.

What is claimed is:

1. A display device, comprising:
a display panel that includes a through hole and a pixel area that surrounds the through hole and includes pixels that display an image;
a pressure sensor disposed on a surface of the display panel and configured to sense an externally applied pressure;
a light-emitting member disposed at a position that overlaps the through hole of the display panel and outputs light through the through hole toward a front side of the display panel; and
a light-receiving sensor that faces the front side of the display panel and is configured to sense light reflected from the front side of the display panel toward the display panel.

2. The display device of claim 1, wherein the pressure sensor includes a first optical hole that overlaps the through hole in a thickness direction of the display panel.

3. The display device of claim 2, wherein a dead space area surrounds the through hole and the light-receiving sensor is disposed in the dead space area adjacent to the through hole of the display panel wherein the light-receiving sensor senses light reflected by a body part or an object from the front side of the display panel.

4. The display device of claim 3, further comprising:
a panel bottom cover that includes a second optical hole located on a rear side of the pressure sensor and aligned with the through hole of the display panel in the thickness direction of the display panel,
wherein the panel bottom cover protects the display panel.

5. The display device of claim 4, further comprising:
a main circuit board,
wherein the main circuit board fixes the light-emitting member so that the light-emitting member is aligned with the through hole of the display panel, and
wherein a main processor is mounted on the main circuit board and is configured to generate a pulse wave signal based on an optical signal according to an amount of light detected by the light-receiving sensor and calculate blood pressure based on the pulse wave signal.

6. The display device of claim 5,
wherein light emitted from the light-emitting emitting member is absorbed by or reflected from a blood vessel of a user's finger or wrist through the first and second optical holes and the through hole, and
wherein the reflected light is sensed by the light-receiving sensor.

7. The display device of claim 5, wherein the light-emitting member comprises at least one of:
a first light-emitting diode that emits infrared light;
a second light-emitting diode that emits green light; and
a third light-emitting diode that emits visible light of a color other than the green light.

8. The display device of claim 5, wherein the light-emitting member comprises two or more of:
a first light-emitting diode that emits infrared light;
a second light-emitting diode that emits green light; and
a third light-emitting diode that emits visible light of a color other than the green light.

9. The display device of claim 5, wherein the main processor controls a plurality of light-emitting diodes in the light-emitting member and simultaneously turns on or turns off the plurality of light-emitting diodes at a predetermined driving period, or sequentially turns on or turns off the plurality of light-emitting diodes at different periods.

10. The display device of claim 5, wherein the main processor controls a plurality of light-emitting diodes in the light-emitting member wherein at least two of the plurality of light-emitting diodes are simultaneously turned on or turned off at a predetermined first driving period and the other of the plurality of light-emitting diodes is sequentially turned on or turned off in a different period from that of the first driving period.

11. The display device of claim 3, wherein the pressure sensor comprises:
a first base substrate and a second base substrate that face each other,
a first pressure sensor electrode disposed on the first base substrate;
a second pressure sensor electrode disposed on the second base substrate; and
a pressure sensing layer that overlaps the first and second pressure sensor electrodes in a thickness direction of the first base substrate.

12. The display device of claim 11,
wherein the first and second pressure sensor electrodes include a transparent conductive material, and
wherein the pressure sensing layer includes a transparent polymer resin wherein the pressure sensor transmits light.

13. A display device comprising:
a display panel that includes a transparent region and a pixel area that surrounds the transparent region and includes pixels that display an image;
a pressure sensor disposed on a surface of the display panel and configured to sense an externally applied pressure;
a light-emitting member disposed at a position that overlaps the transparent region of the display panel and outputs light through the transparent region toward a front side of the display panel; and
a light-receiving sensor that faces the front side of the display panel and is configured to sense light reflected from the front side of the display panel toward an inside of the transparent region.

14. The display device of claim 13, wherein the pressure sensor includes a first optical hole that overlaps the transparent region in a thickness direction of the display panel.

15. The display device of claim 14, wherein a dead space area surrounds the through hole and the light-receiving sensor is formed in the dead space area adjacent to the transparent region of the display panel, wherein the light-receiving sensor senses light that is reflected by a body part or an object on a front side of the transparent region and is incident toward the transparent region.

16. The display device of claim 15, further comprising:
a bracket disposed on a rear side of the pressure sensor and that includes a sensor hole that is aligned with the transparent region of the display panel in the thickness direction of the display panel.

17. The display device of claim 16, wherein the light-receiving sensor is fixed on a main circuit board that fixes the light-emitting member wherein the light-emitting member is aligned with the through hole of the display panel, and is disposed in the sensor hole of the bracket.

18. The display device of claim 15, further comprising:
a panel bottom cover disposed on a rear side of the pressure sensor and that includes a second optical hole that is aligned with the through hole of the display panel in the thickness direction of the display panel,
wherein the panel bottom cover protects the display panel.

19. The display device of claim 18, wherein the light-receiving sensor is fixed on a main circuit board that fixes the light-emitting member wherein the light-emitting member is aligned with the through hole of the display panel, and is disposed in the second optical hole of the panel bottom cover.

20. The display device of claim 16, further comprising a cover window disposed on the front side of the display panel, wherein the light-receiving sensor is integrally formed with or incorporated into the cover window.

21. A method of measuring blood pressure by a display device, comprising:
recognizing, by a touch sensitive display device, a pressure applied to the display device by an object;
detecting the pressure applied to the display device by the object as the pressure increases to a maximum value and then decreases using a pressure sensor;
sensing light reflected by the object using a light-receiving sensor;
generating a pulse wave signal according to the pressure based on a pressure value calculated by the pressure sensor and an optical signal according to an amount of light sensed by the light-receiving sensor, and
calculating a blood pressure based on the pulse wave signal.

22. The method of claim 21, wherein the object is a part of a user's body.

23. The method of claim 21, further comprising, operating the display device in a blood pressure measurement mode, after recognizing that a touch is made,
wherein when a user touches a position that is not a blood pressure measurement position, the display device operates in a touch mode, and
when the user touches a position associated with a blood pressure measurement position, the display device operates in the blood pressure measurement mode.
